(19) United States
(12) Patent Application Publication
(10) Pub. No.: US 2013/0260481 A1
(43) Pub. Date: Oct. 3, 2013

(54) ANALYSIS DEVICE AND ANALYSIS METHOD

(71) Applicants: Yuichiro Shimizu, Osaka-shi (JP); Ikuo Nakano, Osaka-shi (JP); Michinobu Mieda, Osaka-shi (JP)

(72) Inventors: Yuichiro Shimizu, Osaka-shi (JP); Ikuo Nakano, Osaka-shi (JP); Michinobu Mieda, Osaka-shi (JP)

(73) Assignee: SHARP KABUSHIKI KAISHA, Osaka-shi, Osaka (JP)

(21) Appl. No.: 13/993,282

(22) PCT Filed: Nov. 18, 2011
(86) PCT No.: PCT/JP2011/076697

§ 371 Date: Jun. 11, 2013

(30) Foreign Application Priority Data
Dec. 13, 2010 (JP) 2010-277335

(51) Int. Cl. G01N 33/53

(52) U.S. Cl. CPC G01N 33/5302
USPC 436/501; 422/69

(57) ABSTRACT

To provide an analysis device capable of quantitatively measuring the concentration of a substance without diluting a sample containing the substance to be analyzed, detection portions (5) that detect the substance in different concentration ranges are formed in a plurality of minute flow paths (2), respectively, and the concentration of the substance to be detected can be obtained within a calibration range in any one of the detection portions (5).

### TECHNICAL FIELD

The present invention relates to an analysis device and an analysis method, and particularly, to a device detecting and analyzing a specific component (for example, an enzyme, a substrate, a cytokine, or an antibody) contained in blood and a method of detecting and analyzing the specific component.

### BACKGROUND ART

Immunoanalytical methods using an antigen-antibody reaction are useful for analysis and measurement in, for example, the field of medicine, the field of biochemistry, the field of measurement of allergen or the like. However, the immunoanalytical methods according to the related art have problems in that, for example, it takes a long time to perform analysis and an operation is complicated.

In recent years, micro technologies (Micro Electro Mechanical Systems, MEMS) in which minute processing technologies or the like of semiconductor are applied have been developed. In analysis in the field of biochemistry such as protein substances and genes, micro technologies (Micro Total Analytical System, μ-TAS) using an antigen-antibody reaction have rapidly been advanced.

For example, PTL 1 discloses a micro-channel type analysis device that has a substrate in which a minute flow path (hereinafter, also referred to as a “micro-channel”) with a width of a micro-order length is formed on its surface. The analysis device disclosed in PTL 1 analyzes a detection target substance (hereinafter, also referred to as a “target substance”), such as an antigen, using an antibody fixed to the micro-channel or an artificial antibody. A technology for realizing shortening of an analysis time and simplification of an analysis operation using such an analysis device has been suggested.

The configuration of the analysis device disclosed in PTL 1 is illustrated in FIG. 12. In the micro-channel type analysis device, as illustrated in FIG. 12, a micro-channel 201, an injection hole 202 through which a solution is injected into the micro-channel 201, a liquid reserve portion 203 which reserves the solution, and a discharge hole 204 through which the solution is discharged from the analysis device are formed on the surface of a substrate 200 formed of a light transmissive material such as glass or plastic. The injection hole 202 and the discharge hole 204 are each formed in both ends of the micro-channel 201. The liquid reserve portion 203 is connected to the discharge hole 204. An antibody fixing portion 205 is formed inside the micro-channel 201. In the antibody fixing portion 205, an antibody specifically coupled with a target substance in a solution is fixed according to a known fixing method (for example, a method of fixing an antibody using physical adsorption or a method of fixing an antibody by forming covalent bonding between an amino group of the antibody and a functional group of a fixing portion). Here, the antibody refers to a substance that has specific affinity to a target substance.

FIG. 13 is a diagram illustrating a method of analyzing a target substance using the analysis device illustrated in FIG. 12. As illustrated in FIG. 13, a sample containing a target substance 220 is mixed with a solution containing a labeled antibody 223. The labeled antibody 223 is an antibody which is formed when the labeled substance 221 which can optically be detected is bonded with an antibody 222 which can be bonded with the target substance 220. The labeled antibody 223 is bonded with the target substance 220 to form an immune complex (a complex produced by a reaction between the labeled antibody 223 and the target substance 220) 224.

Next, a solution containing the immune complex 224 is injected from the injection hole 202 illustrated in FIG. 12 using an external pump, and then is distributed to the micro-channel 201. When the solution containing the immune complex 224 reaches the antibody fixing portion 205, as illustrated in FIG. 13, the antibody 225 fixed to the antibody fixing portion 205 is bonded with the immune complex 224 in the solution. Thus, in the antibody fixing portion 205, a complex 226 formed from the antibody 225, the target substance 220, and the labeled antibody 223 is formed.

Thereafter, the target substance 220 is detected by optically detecting the labeled substance 221 bonded with the labeled antibody 223 in the complex 226 formed in the antibody fixing portion 205. By detecting light absorption, fluorescence, luminescence, or the like of the labeled substance 221, the detection of the labeled substance 221 is performed, for example, using a predetermined analysis device of ultraviolet-visible spectroscopic analysis, fluorescent analysis, chemiluminescence analysis, thermal lens analysis, or the like according to a kind of labeled substance 221 to be used.

By applying the above-described method of analyzing the target substance to a standard solution containing the target substance 220 with a known concentration, a calibration curve for the concentration of the target substance 220 is generated. The concentration of the target substance 220 in a solution in which the concentration of the target substance 220 is not known can be measured using the calibration curve.

### CITATION LIST

### Patent Literature

PTL 1: Pamphlet of International Publication No. WO 2006/054689 (laid-open on May 26, 2006)

### SUMMARY OF INVENTION

### Technical Problem

A calibration range of the micro-channel type analysis device disclosed in PTL 1 almost depends on the physical property of the antibody 225 fixed to the antibody fixing portion 205. Therefore, when the concentration of the target substance in the sample is higher than the concentration of the target substance which can be bonded with the antibody 225 fixed to the antibody fixing portion 205, the concentration of the target substance may not be accurately determined. Accordingly, it is necessary to dilute the sample so as to fall within the calibration range, before the sample is applied to the analysis device.

Even when the sample is diluted, the target substance in the sample obtained after the dilution may not fall within the calibration range of the analysis device. In this case, it is necessary to further dilute the sample.

When the concentration of the target substance is too high, the concentration of the target substance may not be accurately determined in spite of the fact that the concentration of the target substance falls within the calibration range. This is because when the concentration of the target substance is high, deviation from the primarily approximated calibration curve occurs. Even in this case, in order to accurately determine the concentration of the target substance, it is necessary to dilute the sample.

The content of a solution applied to the micro-channel type analysis device disclosed in PTL 1 is an ultralow volume (a few μL to a few of hundreds of μL). The dilution of such an ultralow volume may be a troublesome task.

Accordingly, in the analysis device according to the related art, there is a problem that a troublesome and complicated dilution operation has to be performed before application of a solution to the analysis device.

The invention has been devised in view of the above-mentioned problems and an object thereof is to provide an analysis device capable of quantitatively measuring the concentration of a target substance in a sample without diluting a solution before application of the solution to the analysis device having a minute flow path and a method of measuring the target substance using the analysis device.

### Solution to Problem

The present invention provides an analysis device including a plurality of first minute flow paths that connect an injection portion accommodating a fluid to be injected to a discharge portion discharging the fluid. The plurality of first minute flow paths are connected to the single injection portion. In each of the plurality of first minute flow paths, a first detection portion is provided between the injection portion and the discharge portion and a first preprocessing portion reducing a concentration of a substance in the fluid is further provided between the injection portion and the first detection portion. A capture substance capturing a substance to be detected is disposed in the first detection portion. A capture substance capturing a substance to be reduced is disposed in the first preprocessing portion. Substances to be detected in the first detection portions of the plurality of first minute flow paths are the same as each other. The first detection portions in the plurality of first minute flow paths detect the substances in different concentration ranges, respectively.

In this configuration, in each first minute flow path, the first preprocessing portion to which the capture substance reducing the concentration of the target substance is fixed is provided on the upstream side of the first detection portion detecting the target substance. Before the sample applied to the injection portion of the analysis device and introduced into each first minute flow path reaches the first detection portion, the sample passes through the first preprocessing portion. Therefore, a part of the target substance in the sample is captured by the capture substance of the first preprocessing portion. Thus, the concentration of the target substance in the sample can be reduced. That is, the concentration of the target substance in the sample is automatically reduced in the analysis device.

The first detection portion detects the target substance in the sample after the sample passes through the preprocessing portion. Each first detection portion detects the target substance in the sample in each different concentration range after the sample passes through the preprocessing portion. That is, the concentration ranges detectable in the first detection portions are different from each other. In the specification, “the concentration range detectable in the detection portion” refers to a concentration range of a concentration of the target substance in the sample which can be quantitatively measured in the detection portion after the sample passes through the preprocessing portion.

Thus, since the first detection portions have the detectable concentration ranges different from each other, the concentration of the target substance in the sample passing through the first preprocessing portion can be included in any one of the detectable concentration ranges. The concentration of the target substance can be accurately determined by measuring the concentration of the target substance using the first detection portion having the detectable concentration range including the concentration of the target substance in the sample.

For example, when the concentration of the target substance in the sample provided in the analysis is not constant in each sample and is included in the concentration range of a to b (μg/mL), the concentration range detectable in a given first detection portion is set to a range of x to b′ (μg/mL) and the concentration range detectable in another first detection portion is set to a range of a′ to y (μg/mL) (where x≦a<a′≦b′<b≦y). Thus, the concentration of the target substance included in the concentration range of x to b′ in the range of a to b can be accurately determined by the given first detection portion described above. Further, the concentration of the target substance included in the concentration range of a′ to y in the range of a to b can be accurately determined by the other first detection portion described above. Accordingly, the concentration of the target substance can be quantitatively measured using the analysis device with the above-described configuration, in spite of the fact that any sample is used as long as the sample is a sample having the target substance with the concentration included in the concentration range of a to b.

Since the first detection portions have the detectable concentration ranges different from each other, the concentration range detectable by one analysis device is expanded compared to a configuration in which one detection portion is provided. Accordingly, the analysis device according to the invention can accurately determine the concentration of the target substance in an expanded concentration range.

In the specification, the “first minute flow path” refers to a minute flow path in which the preprocessing portion and the detection portion are provided. The lengths or shapes of the first minute flow paths may be the same as each other or may be the different from each other.

In the specification, the term, “the sample,” refers to a specimen (material to be detected) applied to the injection portion of the analysis device and may not include an aimed substance (target substance) to be detected.

In the specification, the term, “the capture substance,” refers to a substance that forms covalent bonding or non-covalent bonding with the target substance by specific mutual interaction with the target substance. Specifically, the capture substance is a substance that has a relation of a host and a gate with the target substance. Examples of the capture substance include an antigen, an antibody, an enzyme, a substrate, a ligand, a receptor, DNA, sugar, a peptide, or a synthetic polymer (for example, a molecularly imprinted polymer).

In the specification, the term, “the injection portion,” refers to an entrance into which a sample to be analyzed and a fluid used in the analysis device is injected and may also have a function of storing the sample to be injected in advance. In the specification, the term, “the discharge portion,” refers to an exit in which the analyzed sample and the fluid used in the analysis are discharged from the analysis device and may also have a function of storing the discharged sample and fluid.

In the specification, the terms, “the upstream side” and “the downstream side,” are concepts used with reference to the flow of the fluid in the minute flow path. Unless mentioned otherwise, the direction of the injection portion in the flow path is the “upstream side” and the direction of the discharge portion is the “downstream side.”

The present invention provides an analysis device including first and second minute flow paths that connect an injection portion accommodating a fluid to be injected to a discharge portion discharging the fluid. The first and second minute flow paths are connected to the single injection portion. First and second detection portions are provided in the first and second minute flow paths, respectively. A first preprocessing portion reducing a concentration of a substance in the fluid is further provided in the first minute flow path between the injection portion and the first detection portion. A first capture substance capturing a substance to be detected is disposed in the first detection portion. A second capture substance capturing the substance to be detected is disposed in the second detection portion. A capture substance capturing the substance to be reduced is disposed in the first preprocessing portion. The substances to be detected in the first and second detection portions are the same as each other. The first and second detection portions detect the substances in different concentration ranges, respectively.

In this configuration, the concentration of the target substance in the sample can be determined using any one of the concentration ranges detectable in the first and second detection portions. For example, when the concentration of the target substance in the sample provided in the analysis is not constant in each sample and is included in the concentration range of a to b (μg/mL), the concentration range detectable in the first detection portion can be set to a range of a′ to y (μg/mL) and the concentration range detectable in the second detection portion can be set to a range of x to b′ (μg/mL) (where x≦a<a′≦b′<b≦y). Thus, the concentration of the target substance included in the concentration range of x to b′ in the range of a to b can be accurately determined by the second detection portion. Further, the concentration of the target substance included in the concentration range of a′ to y in the range of a to b can be accurately determined by the first detection portion. Accordingly, the concentration of the target substance can be quantitatively measured using the analysis device with the above-described configuration, in spite of the fact that any sample is used as long as the sample is a sample having the target substance with the concentration included in the concentration range of a to b.

In the specification, the “second minute flow path” refers to a minute flow path in which the preprocessing portion is not provided and the detection portion is provided.

### Advantageous Effects of Invention

According to the present invention, a substance can be analyzed by a plurality of detection portions, even when a sample containing the substance with a concentration considerably exceeding a range which can be analyzed by the detection portions is used. Thus, even when a user does not dilute the sample, the sample can directly be introduced to the analysis device. In addition, quantitative measurement can be realized.

### BRIEF DESCRIPTION OF DRAWINGS

FIGS. 1(a) and 1(b) are plan views illustrating a micro-channel type analysis device according to a first embodiment of the invention.

FIGS. 2(a) and 2(b) are diagrams illustrating one form of one constituent component of the micro-channel type analysis device according to the first embodiment of the invention.

FIGS. 3(a) and 3(b) are diagrams illustrating one form of one constituent component of the micro-channel type analysis device according to the first embodiment of the invention.

FIGS. 4(a) and 4(b) are diagrams illustrating one form of one constituent component of the micro-channel type analysis device according to the first embodiment of the invention.

FIG. 5 is a plan view illustrating another micro-channel type analysis device according to the first embodiment of the invention.

FIG. 6 is a plan view illustrating a micro-channel type analysis device according to a second embodiment of the invention.

FIGS. 7(a) and 7(b) are plan views illustrating a micro-channel type analysis device according to a third embodiment of the invention.

FIG. 8 is a plan view illustrating another micro-channel type analysis device according to the third embodiment of the invention.

FIG. 9 is a plan view illustrating a micro-channel type analysis device according to a fourth embodiment of the invention.

FIG. 10 is a plan view illustrating a micro-channel type analysis device according to the invention.

FIG. 11 is a graph illustrating an analysis result obtained using the micro-channel type analysis device according to the invention.

FIG. 12 is a schematic diagram illustrating a micro-channel type analysis device according to the related art.

FIG. 13 is a schematic diagram illustrating a reaction in an antibody fixing portion formed in the micro-channel type analysis device according to the related art.

### DESCRIPTION OF EMBODIMENTS

Hereinafter, analysis devices according to embodiments of the invention will be described with reference to the drawings. The invention will be described using a micro-channel type analysis device, but the analysis device according to the invention is not limited to the micro-channel type analysis device. For example, a micro-capillary type analysis device is also included in the scope of the invention.

### First Embodiment

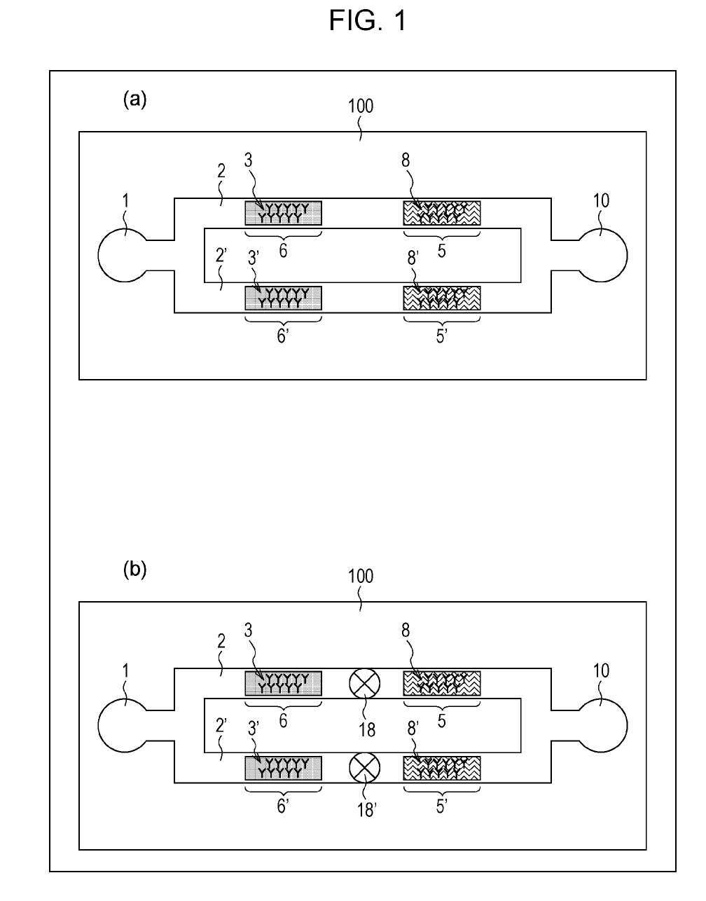

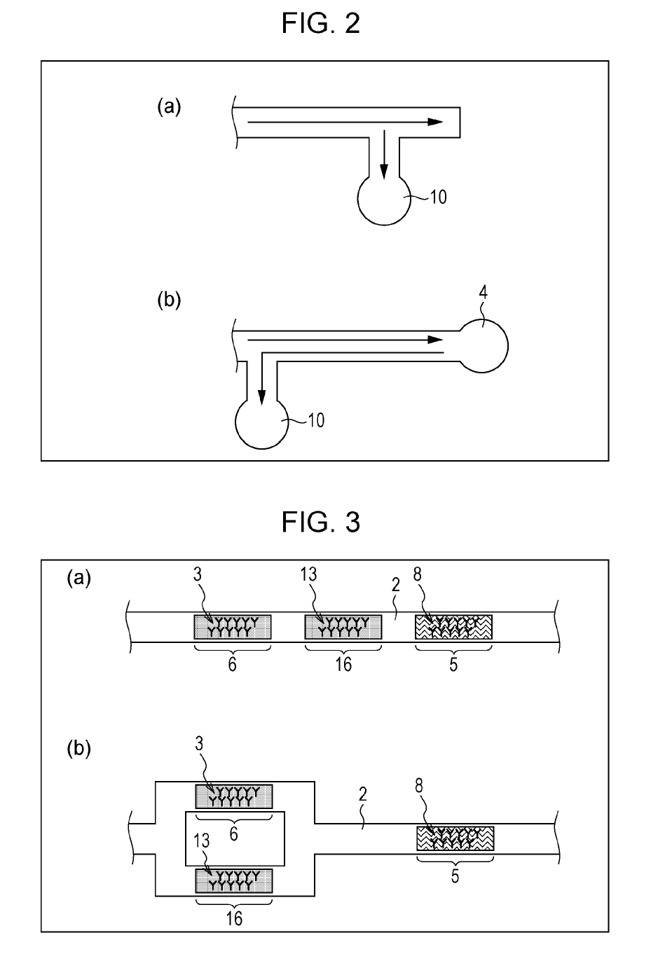

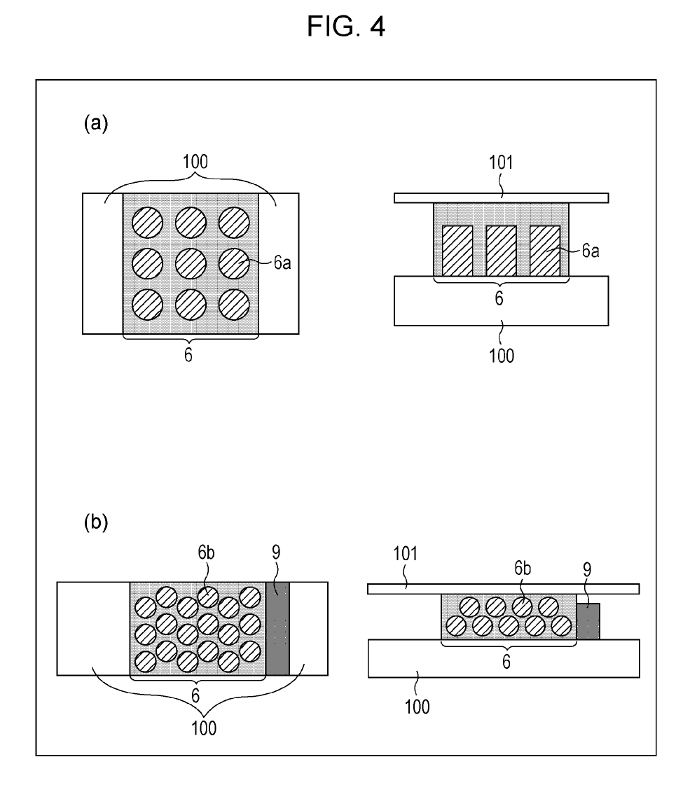

A first embodiment of the invention will be described with reference to FIGS. 1 to 4. FIG. 1 is a plan view illustrating a micro-channel type analysis device according to the first embodiment of the invention. FIGS. 2 and 3 are plan views illustrating one constituent component of the micro-channel type analysis device according to the first embodiment of the invention. FIG. 4 is a plan view (left view) of the one constituent component of the micro-channel type analysis device according to the first embodiment of the invention and a sectional view (right view) when viewed from the lateral side.

### <1. Micro-Channel Type Analysis Device>

The micro-channel type analysis device (micro-channel chip) according to this embodiment includes a substrate 100 and a cover 101 that is superimposed with the substrate 100. Minute grooves (micro-channels) 2 and 2′ of a concave surface are formed on the surface of the substrate 100. In the specification, the phase “minute” means a portion with a diameter of μm order, and specifically, means a portion with a size which can be formed using a minute processing technology of a semiconductor.

The micro-channels 2 and 2′ define flow paths of the analysis device according to this embodiment. The micro-channels 2 and 2′ may be the same as each other or may be different from each other. Specifically, the lengths or shapes of the micro-channels 2 and 2′ may be the same as each other or may be different from each other.

An injection portion 1 that accommodates a fluid to be injected and a discharge portion 10 that discharges a fluid from a flow path are further formed on the surface of the substrate 100 and are connected to both ends of each micro channel 2. That is, the micro-channels 2 are connected to the injection portion 1 and the discharge portion 10 on the surface of the substrate 100. The injection portion 1 can be a portion that stores the fluid to be injected to the micro-channels 2 and the discharge portion 10 can be a portion that stores the fluid to be discharged from the micro-channels 2. In the specification, a boundary portion between the micro-channel 2 and the injection portion 1 or the discharge portion 10 is referred to as an injection hole and a discharge hole (not illustrated), as necessary.

The micro-channel 2′ is a bypath channel that is diverged from the micro-channel 2 and is joined again between the injection portion 1 and the discharge portion 10. The micro-channel 2′ connects the injection portion 1 to the discharge portion 10 with the micro-channel 2 interposed therebetween.

The micro-channels 2 and 2′ are isolated from the outside of the substrate by superimposing the substrate 100 and the cover 101. However, first and second through holes (not illustrated) penetrating the substrate 100 or the cover 101 each cause the injection portion 1 and the discharge portion 10 to communicate with the outside of the substrate. Thus, a fluid can be supplied from the outside of the substrate to the micro-channels 2 and 2′ or a fluid can be discharged from the micro-channel 2 to the outside of the substrate.

Detection portions 5 and 5′ that detects a substance in the fluid flowing in the micro-channels 2 and 2′ are installed inside the micro-channels 2 and 2′. Capture substances 8 and 8′ that capture the same substance to be detected and analyzed are fixed to the detection portions 5 and 5′. As a result, the detection portions 5 and 5′ can detect the same target substance in a sample introduced and supplied from the injection portion 1 to be analyzed.

In the micro-channel 2, a preprocessing portion 6 that reduces the concentration of a target substance (a substance to be detected by the detection portion 5) in the fluid is installed between the injection portion 1 and the detection portion 5. A capture substance 3 that captures the target substance is fixed to the preprocessing portion 6. Likewise, in the micro-channel 2′, a preprocessing portion 6′ that reduces the concentration of a target substance (a substance to be detected by the detection portion 5′) in the fluid is installed between the injection portion 1 and the detection portion 5′. A capture substance 3′ that captures the target substance is fixed to the preprocessing portion 6′.

The detection portions 5 and 5′ each detects the target substance in different concentration ranges. The detection of the target substance in the different concentrations can be realized, for example, by causing detection sensitivities of the detection portions 5 and 5′ to be the same as each other and causing the amount (for example, a molar amount) of the capture substance 3 disposed in the preprocessing portion 6 to be different from the amount (for example, a molar amount) of the capture substance 3′ disposed in the preprocessing portion 6′. In order to cause the detection sensitivities of the detection portions 5 and 5′ to be the same, for example, a capture substance capable of capturing the same molar amount of target substance may be fixed to the detection portions 5 and 5′. In this case, the same capture substance is preferably fixed to the detection portions 5 and 5′ under the same condition (for example, the same molar amount).

The concentration ranges in the detection portions 5 and 5′ preferably overlap with each other at least partially. Thus, the concentration of the target substance in various samples can be measured without omission.

As illustrated in FIG. 1(b), valves 18 and 18′ that regulate a flow direction from the injection portion 1 to the discharge portion 10 or temporally controls the flow of the fluid may be installed inside the micro-channels 2. The valves 18 and 18′ are preferably installed between the preprocessing portion 6 and the detection portion 5 and between the preprocessing portion 6′ and the detection portion 5′. It is not necessary to install both the valves 18 and 18′, but any one of the valves 18 and 18′ may be installed.

In the analysis device according to this embodiment, although not illustrated, driving means accelerating movement of the fluids in the micro-channels 2 and 2′ from the injection portion 1 to the discharge portion 10 may be connected to at least one of the injection portion and the discharge portion. Examples of the driving means include an extrusion pump and an suction pump. When the fluid is sent into the micro-channel 2 or 2′ using the extrusion pump, the extrusion pump may be connected to the injection portion 1. When the fluid is taken out from the micro-channel 2 or 2′ using the suction pump, the suction pump may be connected to the discharge portion 10. As well as using the above-described pumps, the fluid can flow using the capillary phenomenon or a water absorption substance according to the known method of the related art.

The fluid provided to the analysis device according to this embodiment may be a gas or a liquid. However, the fluid may be preferably a liquid, when the fluid is used in a biochemistry analysis according to a micro technology.

Hereinafter, the portions included in the analysis device according to this embodiment will be described in detail.

### <1.1 Substrate>

For example, a substrate with an insulation property can be used as the substrate 100 and the cover 101. Examples of the substrate with the insulation property include a silicon substrate, a quartz substrate, an aluminum oxide substrate, a glass substrate, and a plastic substrate in which a material, such as an oxide film, with an insulation property is formed on its surface. When a substrate is optically detected, a light transmissive substrate can be used as the substrate 100 or the cover 101. Examples of the light transmissive substrate include a glass substrate, a quartz substrate, and a substrate formed of a light transmissive resin. When a substance is detected using chemiluminescence, a glass or plastic material (for example, polyimide, polybenzimidazole, polyether ether ketone, polysulfone, polyetherimide, polyether sulfone, or polyphenylene sulphite) having small spontaneous fluorescence and a light transmissive property can also be used as the substrate 100 or the cover 101. The thickness of the substrate 100 preferably used in the micro-channel type analysis device is in the range of about 0.1 mm to about 5 mm. The thickness of the cover 101 may be the same as the thickness of the substrate 100, may be thinner than the thickness of the substrate 100, or may be thicker than the thickness of the substrate 100.

### <1.2 Micro-Channel, Injection Portion, and Discharge Portion>

The depths of the micro-channels 2 and 2′ formed on the surface of the substrate 100 are preferably in the range of about 0.1 μm to about 1000 μm and the widths thereof are preferably in the range of about 0.1 μm to about 1000 μm, but the invention is not limited thereto. The lengths of the micro-channels 2 and 2′ can be appropriately designed according to the size of the substrate 100 and are preferably in the range of about 50 μm to about 800 μm, but the invention is not limited thereto. The depth, width, and length of the micro-channel 2 may be the same as or may be different from the depth, width, and length of the micro-channel 2′.

The flow paths of the micro-channels 2 and 2′ may have a rectangular cylinder shape or may have a circular cylinder shape according to the flow direction of the fluid. That is, the shapes the cross-sectional surfaces of the micro-channels 2 and 2′ perpendicular to the flow direction of the fluid can be rectangular, trapezoidal, circular (semi-circular). The shape of the micro-channel 2 may be the same as or may be different from the shape of the micro-channel 2′.

The micro-channels 2 and 2′ may be produced, for example, by forming unevenness portions on the substrate 100. For example, concave portions may be formed on the substrate 100 and the concave portions may serve as the micro-channels 2 and 2′. Further, a plurality of convex portions may be formed on the substrate 100 and regions surrounded by the convex portions may serve as the micro-channels 2 and 2′. Furthermore, the concave portions and convex portions may be formed and combinations of the concave portions and the convex portions may be formed to produce the micro-channels 2 and 2′.

Examples of a method of forming the unevenness portions on the substrate 100 include a mechanical processing method as a directly processing method, a laser processing method, and a method of mold injection using a metallic mold, press molding, or casting. The mold injection using a metallic mold is particularly appropriately used since mass production is excellent and shape reproducibility is high. When the material of the substrate 100 is silicon, glass, or the like, a pattern of the micro-channel 2 on the substrate 100 can be formed by a photolithographic method or an etching method.

The method of forming the injection portion 1 and the discharge portion 10 on the substrate 100 in advance has been illustrated. However, the invention is not limited, as long as the micro-channels 2 and 2′ communicate with the outside of the substrate via the injection portion 1 and the discharge portion 10. For example, the injection portion 1 and the discharge portion 10 may be formed as first and second through holes. The sizes of the injection portion 1 and the discharge portion 10 can be appropriately changed according to the sizes and shapes of the micro-channels 2 and 2′. However, the diameters of the injection portion 1 and the discharge portion 10 are preferably equal to or greater than 10 μm, since the analysis device according to this embodiment is used as a micro-channel type analysis device. Further, the injection portion 1 and the discharge portion 10 formed as portions different from the substrate 100 may be disposed in the outside of the substrate and may be configured to be connected to an injection hole and a discharge hole via the first and second through holes, respectively.

In the configuration illustrated in FIG. 1, the injection portion 1 and the discharge portion 10 are connected in both ends of the micro-channels 2. However, portions of the micro-channels 2 in which the injection portion 1 and the discharge portion 10 are connected are not limited to both ends of the micro-channels. Specifically, the injection portion 1 may be connected to the micro-channel 2 on the downstream of the division portion in which the micro-channel 2′ is diverged from the micro-channel 2. Further, the discharge portion 10 may be connected to the micro-channel 2 on the upstream side of the joining portion in which the micro-channel 2′ is joined with the micro-channel 2, or may be connected to the micro-channel 2 between the injection portion 1 and the division portion in which the micro-channel 2′ is diverged from the micro-channel 2.

For example, as illustrated in FIGS. 2(a) and 2(b), the injection portion 1 (not illustrated) may be connected to one end of the micro-channel 2 and the discharge portion 10 may be connected to the micro-channel 2 between the injection portion 1 and the other end of the micro-channel 2. In this case, the other end of the micro-channel 2 may be the blind end, as illustrated in FIG. 2(a) or may be connected to an air hole 4 through which a gas in the micro-channels 2 and 2′ is discharged, as illustrated in FIG. 2(b). In FIGS. 2(a) and 2(b), arrows indicate the flow direction of the fluid.

When the fluid is moved toward the discharge portion 10 in the analysis device configured as in FIG. 2(a), the fluid is divided into a fluid that directly reaches the discharge portion 10 and a fluid that reaches the other end of the micro-channel 2. The fluid that directly reaches the discharge portion 10 is discharged from the discharge portion. The fluid that reaches the other end of the micro-channel 2 flows backward the discharge portion 10 and is discharged from the discharge portion 10, since the other end of the micro-channel 2 is the blind end.

In the analysis device configured as in FIG. 2(b), the discharge portion 10 is connected to the micro-channel 2 between the injection portion 1 (not illustrated) and the above-described division portion, and the air hole 4 is connected to the other end of the micro-channel 2. A sample provided for analysis is injected into the injection portion 1, and then the sample is moved toward the air hole 4 inside the micro-channels 2 and 2′ (not illustrated). While the sample passes through the detection portions 5 and 5′ (not illustrated), the target substance in the sample is captured by the capture substances 8 and 8′ (not illustrated) of the detection portions 5 and 5′. After the sample passes through the detection portions 5 and 5′, a labeled compound used to detect the target substance captured by the capture substances 8 and 8′ of the detection portions 5 and 5′ is injected into the injection portion 1. Then, the labeled compound is moved toward the air hole 4 inside the micro-channels 2 and 2′. When the labeled compound passes through the detection portions 5 and 5′, the target substance captured by the capture substances 8 and 8′ of the detection portions 5 and 5′ reacts to the labeled compound. After the reaction, the sample existing inside the micro-channels 2 and 2′ and the labeled compound are discharged from the discharge portion 10. Further, a substrate used to detect the labeled compound is injected into the injection portion 1 and is moved toward the air hole 4 inside the micro-channels 2 and 2′. Thus, the substrate is caused to react to the labeled compound and detect the target substance. The details of a detection sequence of the target substance are referred to “1.7 Measurement Method” to be described below.

As a method of moving the sample and a reagent toward the air hole 4 inside the micro-channels 2 and 2′, a known method can be used. For example, the sample and the reagent may be moved toward the air hole 4 inside the micro-channels 2 and 2′ by connecting the above-described suction pump to the air hole 4 and sucking a gas from the air hole 4 by the suction pump. Further, the sample and the reagent may be extruded from the injection portion 1 to the air hole 4 by the extrusion pump by connecting the above-described extrusion pump to the injection portion 1. In this case, the gas inside the micro-channels 2 and 2′ is discharged from the air hole 4 with the extrusion of the sample and the reagent.

A known method can be used as a method of discharging the sample and the reagent in the micro-channels 2 and 2′ from the discharge portion 10. For example, the sample and the reagent may be discharged from the discharge portion 10 by connecting a suction pump to the discharge portion 10 and sucking the sample and the reagent from the discharge portion 10 using the suction pump. Further, the sample and the reagent may be discharge from the discharge portion 10 by connecting the air hole 4 to an extrusion pump and injecting a gas from the air hole to the insides of the micro-channels by the extrusion pump.

In order to prevent the sample and the reagent flowing from the injection portion 1 to the air hole 4 via the micro-channels 2 and 2′ from flowing to the discharge portion 10, the above-described valves are preferably provided in the portions in which the discharge portion 10 is connected to the micro-channels 2. By closing the valves during the flow of the sample and the reagent from the injection portion 1 to the air hole 4 via the micro-channels 2 and 2′, it is possible to prevent the sample and the reagent from flowing to the discharge portion 10. After the detection of the target substance ends, the sample and the reagent can be discharged from the discharge portion 10 by opening the valves according to the above-described discharge method.

### <1.3 Detection Portion>

The detection portions 5 and 5′ are portions that detect a substance in a fluid flowing in the micro-channels 2 and 2′, as described above. As illustrated in FIG. 1, the capture substances 8 and 8′ that capture the same substance (hereinafter, also referred to as a target substance) to be detected and analyzed are fixed to the detection portions 5 and 5′, respectively. The capture substances 8 and 8′ may be substances (for example, an antigen, an antibody, an enzyme, a substrate, a ligand, a receptor, DNA, sugar, a peptide, or a synthetic polymer (for example, a molecularly imprinted polymer)) that have a relation of a host and a gate with the target substance. In particular, the antibody or the synthetic polymer is preferably used since its activity is stabled. A known method such as a physical adsorption method, a chemical bonding method, or a covalent bonding method can be appropriately used as the method of fixing the capture substances 8 and 8′. As long as the capture substances 8 and 8′ can capture the same target substance, the capture substances 8 and 8′ may be the same substance, or may be different substances.

The configurations of the detection portions 5 and 5′ are not particularly limited, but can be appropriately determined according to the method of detecting the target substance. When the target substance is optically detected based on absorbance or luminescence (including fluorescence), light transmissive portions of the micro-channels 2 and 2′ may serve as the detection portions 5 and 5′, respectively. In order to simplify the manufacture of the analysis device, the entire substrate 100 or the entire cover 101 may be formed of a light transmissive material such as glass, quartz, or light transmissive resin. In this case, as illustrated, the capture substance 8 may be fixed to the inner wall surface of the micro-channel 2 of the detection portion 5 and the capture substance 8′ may be fixed to the inner wall surface of the micro-channel 2′ of the detection portion 5′.

When the target substance is electrochemically detected, the detection portions 5 and 5′ may include detection means that includes a detection electrode formed inside the micro-channel. The detection electrode may include at least two electrodes, a reference electrode and a working electrode. However, the detection electrode may preferably include three electrodes, a counter electrode in addition to the reference electrode and the working electrode. FIG. 1 illustrates the configuration in which the capture substance 8 is fixed to the inner wall surface of the micro-channel 2 in the detection portion 5 and the capture substance 8′ is fixed to the inner wall surface of the micro-channel 2′ in the detection portion 5′. When the detection electrode is used, the capture substances 8 and 8′ may be fixed to at least the working electrodes.

The reference electrode, the working electrode, and the counter electrode can be formed in each of the micro-channels 2 and 2′ by a minute processing technology using a photolithography technology of the related art. For example, gold, platinum, silver, chrome, titanium, iridium, copper, or carbon can be used as a conductive material of the electrode. In terms of the stability of a reference potential, a silver/silver chloride electrode is used in the reference electrode.

The detection portions 5 and 5′ may have the same configuration or may have different configurations as a configuration for detecting the target substance. That is, the detection portions 5 and 5′ may have the above-described configuration for optically detecting the target substance or the above-described configuration for electrochemically detecting the target substance. The detection portion 5 may have the configuration for optically detecting the target substance and the detection portion 5′ may have the configuration for electrochemically detecting the target substance. The detection portion 5 may have the configuration for electrochemically detecting the target substance and the detection portion 5′ may have the configuration for optically detecting the target substance. At least one of the detection portions 5 and 5′ may have both the configuration for optically detecting the target substance and the configuration for electrochemically detecting the target substance.

### <1.4 Preprocessing Portion>

As described above, the preprocessing portion 6 is a portion that reduce the concentration of a substance (a substance to be detected by the detection portion 5) in a fluid. As illustrated in FIG. 1, the capture substance 3 that captures the target substance is fixed to the preprocessing portion 6 installed between the injection portion 1 and the detection portion 5. Likewise, the preprocessing portion 6′ is a portion that reduces the concentration of a substance (a substance to be detected by the detection portion 5′) in a fluid. The capture substance 3′ that captures the target substance is fixed to the preprocessing portion 6 installed between the injection portion 1 and the detection portion 5′.

As in the capture substances 8 and 8′, the capture substances 3 and 3′ may be substances (for example, an antigen, an antibody, an enzyme, a substrate, a ligand, a receptor, a DNA, a sugar, a peptide, or a synthetic polymer (for example, a molecularly imprinted polymer)) that has a relation of a host and a gate with the target substance. In particular, the antibody or the synthetic polymer is preferably used since its activity is stabled. The capture substances 3 and 3′ may be the same substance or may be different substances, as long as the capture substances 3 and 3′ can capture the same target substance. The capture substances 3 and 3′ and the capture substances 8 and 8′ are preferably the same substance. By using the capture substances with the same characteristics, it is possible to improve the productivity of the analysis device according to this embodiment or reduce the manufacturing cost and it is also possible to improve development efficiency. A known method such as a physical adsorption method, a chemical bonding method, or a covalent bonding method can be appropriately used as the method of fixing the capture substances 3 and 3′.

As illustrated in FIG. 3, in the analysis device according to this embodiment, the plurality of preprocessing portions 6 may be installed in the micro-channel 2. Specifically, as illustrated in FIG. 3(a), two or more preprocessing portions 6 and 16 may be disposed in series in a single flow path. As illustrated in FIG. 3(b), the preprocessing portions 6 and 16 may be disposed in a plurality of flow paths diverged from a single flow path and joined with each other again, respectively. The capture substance 13 is fixed to the preprocessing portion 16. The capture substance 13 is preferably the same as the capture substance 3. Likewise, the plurality of preprocessing portions 6′ may be installed in the micro-channel 2′.

The configuration of the preprocessing portion 6 is not particularly limited. For example, as illustrated in FIG. 1, the capture substance 3 may be fixed to the inner wall surface of the micro-channel 2 of the preprocessing portion 6. Likewise, the configuration of the preprocessing portion 6′ is not particularly limited either. For example, as illustrated in FIG. 1, the capture substance 3′ may be fixed to the inner wall surface of the micro-channel 2′ of the preprocessing portion 6′. In order to expand an area in which the capture substances 3 and 3′ are fixed, a three-dimensional structure may be disposed in each of the micro-channel 2 of the preprocessing portion 6 and the micro-channel 2′ of the preprocessing portion 6′. When such a three-dimensional structure is provided, the molar amounts of the capture substances 3 and 3′ fixed to the preprocessing portions 6 and 6′ increase. As a result, reaction efficiency between the target substance and the capture substances 3 and 3′ is improved, and thus the concentration of the target substance can be reduced very efficiently.

Examples of the structure include a pillar structure 6a (illustrated in FIG. 4(a)), a porous structure (not illustrated), a fine particle 6b illustrated in FIG. 4(b). As illustrated in FIG. 4(b), a dam cutoff portion 9 that blocks movement of the fine particles 6b is provided in the micro-channel 2 between the injection portion 1 and the detection portion 5 or in the micro-channel 2′ between the injection portion 1 and the detection portion 5′. Thus, when a solution containing the fine particles 6b is introduced from the injection portion 1, the fine particles 6b can be caused to remain by the dammed portion 9. Sets of the fine particles 6b remaining by the dammed portion 9 form the preprocessing portions 6 and 6′. The dammed portion 9 is not particularly limited, as long as the dammed portion 9 is configured to inhibit pass of the fine particles 6b without interference of flow of a fluid.

At least one of the preprocessing portions 6 and 6′ preferably includes additional detection means. That is, before the detection portions 5 and 5′ detect the target substance, the preprocessing portions 6 and 6′ preferably detect the target substance. When the detection means is included in the preprocessing portions 6 and 6′, it is possible to confirm whether the target substance is captured in the preprocessing portions 6 and 6′. In this case, when the capture of a given amount of target substance in the preprocessing portions 6 and 6′ may not be confirmed due to deterioration of the capture substances 3 and 3′ or another abnormality, the analysis failure (analysis error) can be determined. That is, the analysis device according to this embodiment may further include a determination portion that determines whether the target substance is present in the preprocessing portions 6 and 6′. An amount of target substance captured in the preprocessing portions 6 and 6′ and determined as the analysis failure is not particularly limited and can be appropriately set by those skilled in the art.

The detection means provided in the preprocessing portions 6 and 6′ is preferably the same as the detection means provided in the detection portions 5 and 5′, but may be different from the detection means provided in the detection portions 5 and 5′. The details of the detection means are referred to “1.3 Detection Portion” described above.

### <1.5 Valve Structure>

The valves 18 and 18′ have, for example, a structure for regulating the flow direction of the fluid in the micro-channels 2 and 2′, a structure for physically stopping the flow of the fluid in the micro-channels 2 and 2′, a structure for cutting off the fluid in the micro-channels 2 and 2′, a structure for separating the fluid in the micro-channels 2 and 2′, respectively, and may have all of the functions, as necessary. Preferable examples of the valves 18 and 18′ include a turning screw type value, a free inflow and outflow damp plate, a closure by pressure, and a liquid disconnection by gas control. By providing the valves 18 and 18′, the reaction time in the preprocessing portions 6 and 6′ and/or the reaction time in the detection portions 5 and 5′ can be prolonged.

### <1.6 Method of Adjusting Capture Substance>

In order to quantitatively measure the concentration of the target substance in the detection portions 5 and 5′, it is necessary to adjust the amounts (molar amounts) of the capture substances 3 and 3′ so that the concentration of the target substance is obtained within the concentration range detectable in the detection portions 5 and 5′. That is, the amounts of the capture substances 3 and 3′ are adjusted according to the concentration of the target substance. Since the amounts of the capture substances 3 and 3′ depend not only on the characteristics of the capture substances 3 and 3′ and the capture substances 8 and 8′ but also on the shapes of the micro-channels 2 and 2′, it is necessary to appropriately adjust the amounts of the capture substances 3 and 3′ according to the configuration of the analysis device. An example of the method of adjusting a fixed concentration will be described below.

### (1) Confirmation of Detectable Concentration Ranges

In an analysis device X having the same configuration as the configuration of this embodiment other than a configuration in which the preprocessing portions 6 and 6′ are not provided, concentration ranges detectable by the detection portions 5 and 5′ are inspected using a target substance (standard substance) with a known concentration. In the concentration ranges, the detection result of the target substance in the detection portions 5 and 5′ and the concentration of the target substance preferably have linearity.

The molar amounts of the capture substances 8 and 8′ fixed to the detection portions 5 and 5′ can be determined through such an operation.

### (2) Examination of Condition of Preprocessing Portions 6 and 6′

Capture substances of various concentrations (100, 10, 1, 0.1, and 0.01 μg/mL) are prepared and fixed to the preprocessing portion 6 of the micro-channel 2 and the preprocessing portion 6′ of the micro-channel 2′ in the analysis device according to this embodiment. Next, the detectable concentration ranges are inspected using the standard substance. In this concentration range, the detection result of the target substance in the detection portions 5 and 5′ and the concentration of the target substance preferably have linearity.

### (3) Determination of Condition of Preprocessing Portions 6 and 6′

The concentrations of the capture substances 3 and 3′ indicating a condition close to desired concentration ranges are selected based on the result of the operation (2). At this time, the concentration of the capture substance 3 different from the concentration of the capture substance 3′ is selected. Thus, the detection portions 5 and 5′ can detect the target substance in the different concentration ranges. The fixed condition of the preprocessing portions is determined by performing the operation (2) again using the capture substance with a concentration near the selected concentration.

For example, when the concentration of the target substance in the sample provided in the analysis is included in the concentration range of a to b (μg/mL), the fixed condition of the preprocessing portion is selected so that the concentration range detectable by the detection portion 5 is a range of x to b′ (μg/mL) and the concentration range detectable by the detection portion 5′ is a range of a′ to y (μg/mL) (where x≦a<a′≦b′<b≦y). A relation of “x=a” may be satisfied, but a relation of “x<a” is more preferable. A relation of “a′=b′” may be satisfied, but a relation of “a′<b′” is more preferable. A relation of “b=y” may be satisfied, but a relation of “b<y” is more preferable. In order to realize the concentration ranges, for example, the capture substance may be fixed to each preprocessing portion using the same capture substance so that the amount of capture substance fixed to the preprocessing portion 6 is less than the amount of capture substance fixed to the preprocessing portion 6′. That is, the amounts of capture substances disposed on the respective preprocessing portions satisfy a relation of “the amount of capture substance of the preprocessing portion 6<the amount of capture substance of the preprocessing portion 6′.”

In the above-described sequence, the fixed condition of the capture substances 3 and 3′ and the fixed condition of the capture substances 8 and 8′ are determined. The operation (3) can be performed more simply by causing the concentration ranges (that is, the detection sensitivities of the detection portions 5 and 5′) detectable by the detection portions 5 and 5′ to be same as each other. In order to cause the detection sensitivities of the detection portions 5 and 5′ to be the same as each other, the capture substances that can capture the same molar amount of target substance may be fixed to the detection portions 5 and 5′ (for example, the capture substances and the fixed conditions (molar amounts) of the respective detection portions may be the same as each other). Whether the detection sensitivities of the detection portions 5 and 5′ are the same as each other can be confirmed by the operation (1).

The concentration ranges very suitable for the detection can be adjusted by expanding the areas of the preprocessing portions without preparation of the capture substances 3 and 3′ with various concentrations. For example, as illustrated in FIG. 3(a), the plurality of preprocessing portions may be provided. The concentration can be efficiently reduced down to a lower concentration by passing the sample containing the target substance and provided in the analysis through the preprocessing portions a plurality of times. As illustrated in FIG. 3(b), at least one of the micro-channels 2 and 2′ may be diverged and a plurality of preprocessing portions may be provided in parallel in the diverged micro-channels. When the plurality of preprocessing portions are provided in parallel, the concentration of the sample containing the target substance and provided in the analysis can efficiently be reduced in a shorter time. Therefore, these methods may be combined and used.

Thus, those skilled in the art can appropriately adjust the molar amounts of the capture substances 3 and 3′ in the preprocessing portions, the molar amount of the target substance of the sample provided in the analysis, and the molar amounts of the capture substances 8 and 8′ in the detection portions 5 and 5′.

### <1.7 Measurement Method>

An example of an analysis method of using the analysis device according to this embodiment will be described below. Driving means for causing a fluid to flow in the micro-channels 2 and 2′ may be any one of a method of using an extrusion pump connected to the injection portion 1, a method of using a suction pump connected to the discharge portion 10, and a method of using a capillary force and/or an absorption substance.

### (1) Blocking

A non-specific adsorption inhibitor is introduced from the injection portion 1 to fill the insides of the micro-channels 2 and 2′ so that a substance (non-target substance) which is not a detection target in the sample provided in the analysis is inhibited from being non-specifically adsorbed to the micro-channels 2 and 2′, the preprocessing portions 6 and 6′, and the detection portions 5 and 5′. Next, the non-specific adsorption inhibitor is discharged from the discharge portion 10. A solution of detergent is introduced from the injection portion 1, passes through the micro-channels 2 and 2′, and is discharged from the discharge portion 10. Thus, the extra non-specific adsorption inhibitor remaining in the micro-channels 2 and 2′ is removed. An example of the very suitable non-specific absorption inhibitor includes Protein-Free (Thermo Co., Ltd).

### (2) Introduction of Sample Provided in Analysis

The sample provided in the analysis is introduced from the injection portion 1 to the inside of the micro-channels 2 and 2′. The sample provided in the analysis is moved in the micro-channels 2 and 2′ and is delivered to the preprocessing portions 6 and 6′. While the sample provided in the analysis passes through the preprocessing portions 6 and 6′, the target substance in the sample provided in the analysis is bonded with the capture substances 3 and 3′ of the preprocessing portions 6 and 6′ to be captured in the preprocessing portions 6 and 6′. Thus, the concentration of the target substance in the sample provided in the analysis and passing through the preprocessing portions 6 and 6′ is reduced. At this time, the valves 18 and 18′ are preferably closed so that the target substance in the sample provided in the analysis is sufficiently bonded with the capture substances 3 and 3′ of the preprocessing portions 6 and 6′.

Next, by opening the valves 18 and 18′, as necessary, the sample provided in the analysis is further moved in the micro-channels 2 and 2′ and is delivered to the detection portions 5 and 5′. While the sample provided in the analysis passes through the detection portions 5 and 5′, the target substance in the sample provided in the analysis is bonded with the capture substances 8 and 8′ of the detection portions 5 and 5′ to be captured in the detection portions 5 and 5′. Next, a solution of detergent is introduced from the injection portion 1, is moved in the micro-channels 2 and 2′, and is discharged from the discharge portion 10. Thus, the extra sample provided in the analysis and remaining in the micro-channels 2 and 2′ is removed.

The sample provided in the analysis in the micro-channels 2 and 2′ from the injection portion 1 to the discharge portion 10 may be continuously moved or may be intermittently moved. When the sample provided in the analysis is intermittently moved, for example, the sample provided in the analysis may be retained (incubated) in the regions of the preprocessing portions 6 and 6′ and/or the detection portions 5 and 5′ for a predetermined time. Thus, it is possible to optimize the reaction time of the target substance in the sample provided in the analysis and the capture substances 3 and 3′ and/or the capture substances 8 and 8′.

### (3) Labeling of Target Substance Captured in Detection Portions

A labeled compound which can be bonded with the target substance is introduced from the injection portion 1 to the insides of the micro-channels 2 and 2′ and is delivered to the detection portions 5 and 5′. While the labeled compound passes through the detection portions 5 and 5′, the labeled compound is bonded with the target substance captured in the detection portions 5 and 5′. Through this operation, the target substance captured in the detection portions 5 and 5′ is labeled.

### (4) Detection of Target Substance

By detecting the labeled compound using the detection means, it is possible to detect the target substance in the detection portions 5 and 5′. For example, a fluorescent labeled antibody or an enzyme labeled antibody can be used as the labeled compound, but an antibody different from the capture substances 8 and 8′ is preferably used.

### (4-1) Detection of Target Substance Using Fluorescent Labeled Antibody

When a fluorescent labeled antibody is used in (3) above, the target substance can be detected by directly observing the fluorescence of the detection portions 5 and 5′.

### (4-2) Detection of Target Substance Using Enzyme Labeled Antibody

When an enzyme labeled antibody is used in (3) above, the enzyme labeled antibody is bonded with the target substance, and then a substrate solution for the enzyme is introduced from the injection portion 1 to the micro-channels 2 and 2′. While the substrate solution passes through the detection portions 5 and 5′, the enzyme labeled antibody bonded with the target substance reacts with the substrate solution. The target substance can be detected by detecting a signal obtained from the reaction result according to a known method. Those skilled in the art can appropriately select such a method according to a kind of substrate to be used.

For example, when a substrate emitting fluorescence by the above-described reaction is used, the target substance can be detected by directly observing the fluorescence of the detection portions 5 and 5′. When a substrate of which absorbance varies by the above-described reaction is used, the target substance can be detected by measuring the absorbance of the detection portions 5 and 5′. When a substrate of which electrochemical activity varies by the above-described reaction is used, the target substance can be detected by electrochemical means using an electrode.

The measurement method according to this embodiment is very suitable for the biochemical analysis. The sample to be used is not particularly limited, but blood is preferable in consideration of frequency used in the biochemical analysis. By providing blood according to the above-described measurement method, for example, a blood component such as immune globulin, albumin, GOT, GTP, γ-GPT, HDL, LDL, neutral fat, hemoglobin A1C, uric acid, glucose, adiponectin, leptin, resistin, or TNF-α can be analyzed as the target substance.

The content of the sample used in the micro technology is very small. When a complicated dilution operation is included at the time of preparing a small amount of sample, an error occurs in every preparation. Therefore, it is difficult to perform accurate analysis, and thus reproducibility and/or reliability of the analysis may deteriorate. When the analysis device according to this embodiment is used, the sample dilution operation can be omitted. Therefore, it is possible to improve the reproducibility and/or reliability of the analysis.

In particular, since a danger of affection to an infection disease is involved in order to operate blood, a close attention is necessary in the treatment. By simplifying the process of preparing the sample, such a danger can be reduced. When the analysis device according to this embodiment is used, the sample dilution operation can be omitted. Therefore, the user can treat the blood sample more safely and easily.

When the labeled compound and the substrate solution are introduced from the injection portion 1 to the insides of the micro-channels 2 and 2′, the labeled compound and the substrate solution pass through the preprocessing portions 6 and 6′ before reaching the detection portions 5 and 5′. While the labeled compound and the substrate solution pass through the preprocessing portions 6 and 6′, the labeled compound and the substrate solution can be bonded with and/or react with the target substance captured in the preprocessing portions 6 and 6′. Therefore, the signal can be generated by this reaction. There is sufficiently a probability that this signal may trouble the detection of the target substance in the detection portions. In order to avoid this probability, the substrate solution may be injected from the discharge portion 10 by bonding the captured substances 8 and 8′ with the labeled compound in the detection portions 5 and 5′, and then changing the flow direction in the micro-channels 2 and 2′.

### <1.8 Measurement Result>

For example, a case will be described in which the concentration of the target substance in the sample provided in the analysis is included in the concentration range of a to b (μg/mL), the concentration range detectable in the detection portion 5 is a range of x to b′ (μg/mL), and the concentration range quantitatively detectable in the detection portion 5′ is a range of a′ to y (μg/mL) (where x≦a<a′≦b′<b≦y).

(1) When the concentration of the target substance in the sample provided in the analysis is included in the concentration range equal to or greater than a and less than a′ (μg/mL), the concentration range is within the concentration range detectable in the detection portion 5. Therefore, the detection result of the target substance in the detection portion 5 is within a calibration range of the detection portion 5. On the other hand, since the upper limit of the concentration range equal to or greater than a and less than a′ (μg/mL) is less than the lower limit of the concentration range detectable in the detection portion 5′, the detection result of the target substance in the detection portion 5′ is less than the lower limit of a calibration range of the detection portion 5′.

(2) When the concentration of the target substance in the sample provided in the analysis is included in the concentration range equal to or greater than b′ and equal to or less than b (μg/mL), the lower limit of this concentration range is greater than the upper limit of the concentration range detectable in the detection portion 5. Therefore, the detection result of the target substance in the detection portion 5 is greater than the calibration range of the detection portion 5. On the other hand, since the concentration range equal to or greater than b′ and equal to or less than b (μg/mL) is within the concentration range detectable in the detection portion 5′, the detection result of the target substance in the detection portion 5′ is within the calibration range of the detection portion 5′.

(3) When the concentration of the target substance in the sample provided in the analysis is included in the concentration range equal to or greater than a′ and equal to or less than b′ (μg/mL), this concentration range is within the concentration range detectable in the detection portions 5 and 5′. Therefore, the detection result of the target substance in the detection portions 5 and 5′ is within the calibration range of the detection portions 5 and 5′.

As described above, the detection result of the target substance is within the calibration range of one of the detection portions in (1) and (2) and is out of the calibration range of the other detection portion. In this case, the concentration of the target substance determined using the detection result out of the calibration range may be determined erroneously, and the concentration of the target substance determined using the detection result within the calibration range can be determined correctly. Thus, it is possible to accurately determine the concentration of the target substance in the sample provided in the analysis based on the detection result of each detection portion and the calibration range of each detection portion. In (3), the detection result of the target substance is within the calibration range in both the detection portions. In this case, using at least one of the detection results, it is possible to accurately determine the concentration of the target substance in the sample provided in the analysis.

In (1) to (3), whether an analysis error occurs can be confirmed by comparing the detection results of all the detection portions to each other. That is, in (1) and (2), as described above, the detection result of the target substance is within the calibration range in one of the detection portions and is out of the calibration range in the other detection portion. Accordingly, in (1) and (2), when the detection result of both the detection portions is within the calibration range or when the detection result of both the detection portions is out of the calibration range, it can be confirmed that the detection fails in at least one of the detection portions (an analysis error occurs). As the case in which the detection is considered to fail, in (1), a case can be exemplified in which even when the detection result in the detection portion 5 is within the calibration range of the detection portion 5, the detection result of the detection portion 5′ is not less than the lower limit of the calibration range of the detection portion 5′. In (2), a case can be exemplified in which even when the detection result of the detection portion 5′ is within the calibration range of the detection portion 5′, the detection result of the detection portion 5 is not greater than the upper limit of the calibration range of the detection portion 5.

Further, in (3), when the detection results of both the detection portions are different or when the concentrations calculated from the detection results of both the detection portions are different, it can be confirmed that the detection fails in at least one of the detection portions (an analysis error occurs).

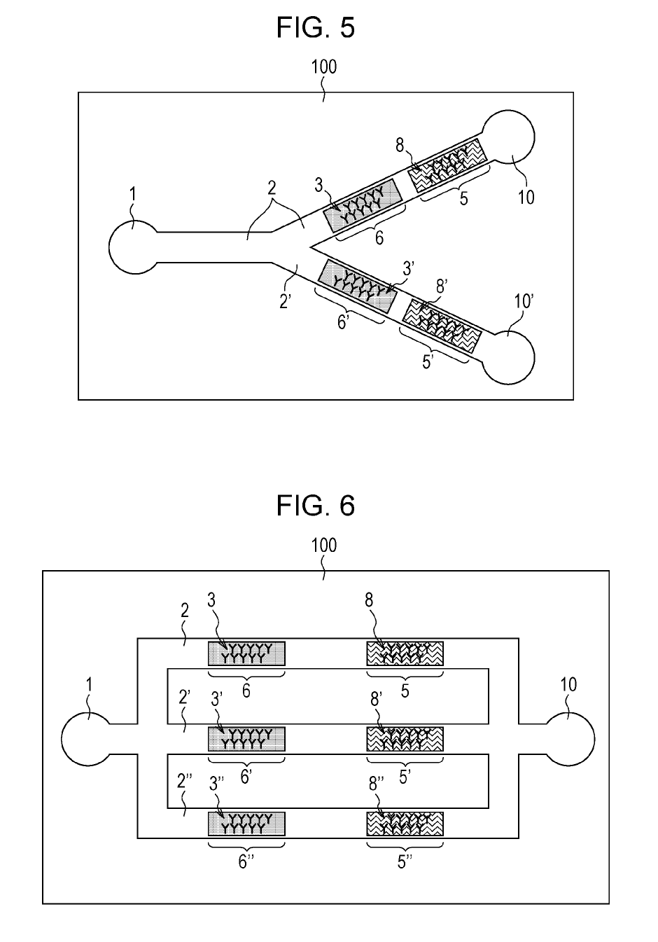

The configuration in which the single discharge portion is shared by the plurality of micro-channels has illustrated in FIG. 1. As illustrated in FIG. 5, each of the plurality of micro-channels may be connected to each independent discharge portion. That is, in the micro-channel type analysis device according to this embodiment, as illustrated in FIG. 5, the micro-channel 2 connecting the injection portion 1 to the discharge portion 10 formed on the surface of the substrate 100 and the micro-channel 2′ connecting the injection portion 1 to a discharge portion 10′ may be formed on the surface of the substrate 100.

The micro-channel 2′ is a flow path that is formed when the micro-channel 2 is diverged between the injection portion 1 and the discharge portion 10. The detection portions 5 and 5′ that detect a substance in the fluid flowing in the micro-channels 2 and 2′ are provided inside the micro-channels 2 and 2′. The capture substances 8 and 8′ that capture the substance to be detected and analyzed are fixed to the detection portions 5 and 5′, respectively. As a result, the target substance in the sample provided in the analysis and introduced from the injection portion 1 can be detected by each of the detection portions 5 and 5′.

The preprocessing portion 6 is provided inside the micro-channel 2 between the detection portion 5 and a division portion in which the micro-channel 2′ is diverged from the micro-channel 2. The preprocessing portion 6′ is provided inside the micro-channel 2′ between the division portion and the detection portion 5′. The capture substances 3 and 3′ that capture the target substance are fixed to the preprocessing portions 6 and 6′, respectively.

When driving means is applied to the micro-channel type analysis device illustrated in FIG. 5, the driving means may be connected to at least one of the injection portion 1 and the discharge portions 10 and 10′.

Thus, the analysis device according to this embodiment is configured to accurately determine the concentration of the target substance in the sample provided in the analysis. When the configuration according to this embodiment is used, the target substance can be measured without diluting the sample provided in the analysis.

### Second Embodiment

As the specific configuration of the analysis device according to the first embodiment, the configuration in which the single bypass channel (the micro-channel 2′) is diverged from the micro-channel 2 which is the main flow path has been illustrated in FIGS. 1 and 5. However, the number of bypass channels is not particularly limited. Each bypass channel has a different detection range. Therefore, when the number of bypass channels increases, the detection range of the analysis device can be expanded.

As a micro-channel type analysis device according to a second embodiment, for example, an analysis device including three micro-channels can be exemplified, as illustrated in FIG. 6. In the analysis device, as illustrated in FIG. 6, three micro-channels 2, 2′, and 2″ are formed on the surface of a substrate 100. The micro-channel 2 connects the injection portion 1 to a discharge portion 10 on the surface of the substrate 100. The micro-channels 2′ and 2″ are bypass channels from which the micro-channel 2 is diverged and joined again between the injection portion 1 and the discharge portion 10. As illustrated in FIG. 6, the micro-channels 2′ and 2″ are diverged in the same division portion in the micro-channel 2 and are joined in the same joining portion.

Detection portions 5, 5′, and 5″ are provided inside the micro-channels 2, 2′, and 2″, respectively. Capture substances 8, 8′, and 8″ that capture the same target substance are fixed to the detection portions 5, 5′, and 5′. Preprocessing portions 6, 6′, and 6″ are provided inside the micro-channels 2, 2′, and 2″ between the injection portion 1 and the detection portions 5, 5′, and 5″, respectively. Capture substances 3, 3′, and 3″ that capture the same target substance are fixed to the preprocessing portions 6, 6′, and 6″.

In the analysis device according to this embodiment, although not illustrated, a value that regulates a flow direction of a fluid in the micro-channels 2, 2′, and 2″ from the injection portion 1 to the discharge portion 10 or controls flow of the fluid may be provided inside each micro-channel.

In the analysis device according to this embodiment, although not illustrated, driving means for accelerating movement of the fluid in the micro-channels 2, 2′, and 2″ from the injection portion 1 to the discharge portion 10 may be connected to at least one of the injection portion 1 and the discharge portion 10.

The description of the method of adjusting the capture substance in the above-described first embodiment can be appropriately changed and applied to a method of adjusting the capture substance in this embodiment. For example, when the concentration of the target substance in the sample provided in the analysis is included in the concentration range of a to b (μg/mL), the fixing condition of the preprocessing portions may be selected so that the concentration range detectable in the detection portion 5 is a range of a₁ to b₁ (μg/mL), the concentration range detectable in the detection portion 5′ is a range of a₂ to b₂ (μg/mL), and the concentration range detectable in the detection portion 5″ is a range of a₃ to b₃ (μg/mL) (where a₁≦a<a₂≦b₁<a₃≦b₂<b≦b₃). A relation of “a₁=a” may be satisfied, but a relation of “a₁<a” is more preferable. A relation of “a₂=b₁” may be satisfied, but a relation of “a₂<b₁” is more preferable. A relation of “a₃=b₂” may be satisfied, but a relation of “a₃<b₂” is more preferable. A relation of “b=b₃” may be satisfied, but a relation of “b<b₃” is more preferable.

In order to realize these concentration ranges, for example, the capture substance of each preprocessing portion may be fixed using the same capture substance so that the amount of capture substance fixed to the preprocessing portion 6 is less than the amount of capture substance fixed to the preprocessing portion 6′ and the amount of capture substance fixed to the preprocessing portion 6′ is less than the amount of capture substance fixed to the preprocessing portions 6″. That is, the amounts of capture substances disposed in the respective preprocessing portions satisfy a relation of “the amount of capture substance of the preprocessing portion 6<the amount of capture substance of the preprocessing portion 6′<the amount of capture substance of preprocessing portion 6″.”

When the number of micro-channels is not three but n, the fixing condition of the preprocessing portions may be selected so that the concentration range detectable in the detection portion 5 in the first micro-channel is a range of a₁ to b₁ (μg/mL), the concentration range detectable in the detection portion 5 in an m-th micro-channel is a range of aₘ to bₘ (μg/mL), and the concentration range detectable in an n-th detection portion 5 is a range of aₙ to bₙ (μg/mL) (where a₁≦a<aₘ≦b₁<aₙ≦bₘ<b≦bₙ).

In order to realize these concentration ranges, for example, the capture substance of each preprocessing portion may be fixed using the same capture substance so that the amount of capture substance fixed to the preprocessing portion 6 in the first micro-channel is less than the amount of capture substance fixed to the preprocessing portion 6 in the m-th micro-channel and the amount of capture substance fixed to the preprocessing portion 6 in the m-th micro-channel is less than the amount of capture substance fixed to the preprocessing portions 6 in the n-th micro-channel. That is, the amounts of capture substances disposed in the respective preprocessing portions satisfy a relation of “the amount of capture substance of the preprocessing portion 6 in the first micro-channel<the amount of capture substance of the preprocessing portion 6 in the m-th micro-channel<the amount of capture substance of preprocessing portion 6 in the n-th micro-channel.”

The configurations of the main constituents such as the substrate, the micro-channels, the injection portion, the discharge portion, the preprocessing portion, the detection portion, the valve structure, the driving means, and the like in the analysis device according to this embodiment are the same as those of the above-described first embodiment. Further, those skilled in the art who read the specification can appropriately modify the configuration of the first embodiment and also apply the measurement method and the measurement result to this embodiment.

### Third Embodiment

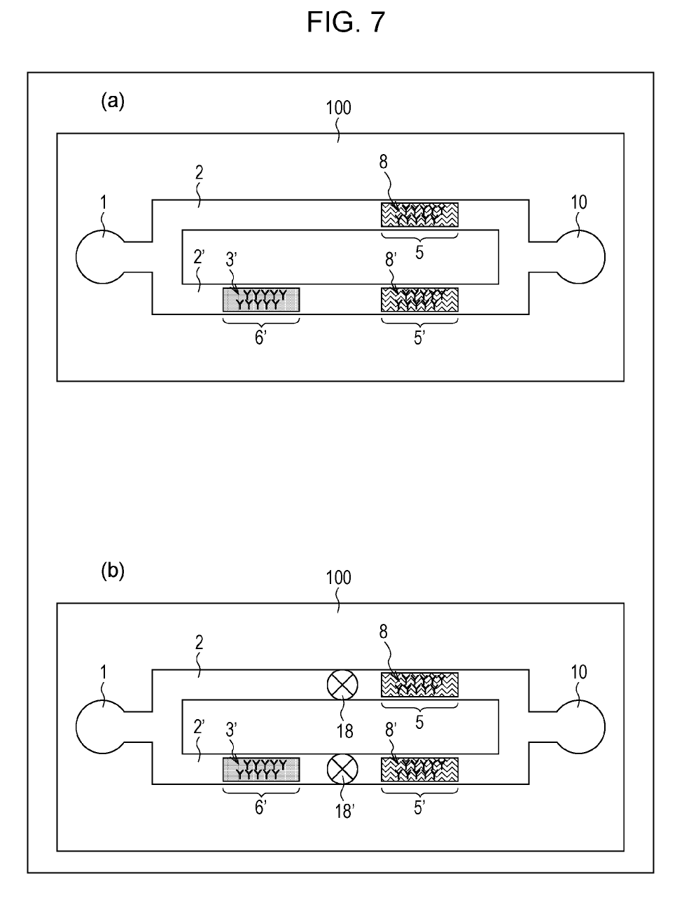

A third embodiment of the invention will be described with reference to FIG. 7. FIG. 7 is a plan view illustrating a micro-channel type analysis device according to the third embodiment of the invention.

In the micro-channel type analysis device according to this embodiment, as illustrated in FIG. 7, micro-channels 2 and 2′ that connect an injection portion 1 to a discharge portion 10 formed on the surface of a substrate 100 are formed on the surface of the substrate 100. The micro-channel 2′ is a bypass channel from which the micro-channel 2 is diverged and joined again between the injection portion 1 and the discharge portion 10. Detection portions 5 and 5′ are provided in the micro-channels 2 and 2′. Capture substances 8 and 8′ are fixed to the detection portions 5 and 5′, respectively. A preprocessing portion 6′ is provided in the micro-channel 2′ between the injection portion 1 and the detection portion 5′. A capture substance 3′ is fixed to the preprocessing portion 6′.

Thus, the analysis device according to this embodiment includes the micro-channel in which the preprocessing portion and the detection portion are provided and the micro-channel in which the preprocessing portion is not provided and the detection portion is provided.

In the analysis device according to this embodiment, a method of adjusting the capture substance may be performed, for example, as follows.

### (1) Confirmation of Detectable Concentration Ranges

In an analysis device X having the same configuration as the configuration of this embodiment other than the configuration in which the preprocessing portion 6′ is not provided, the concentration ranges detectable in the detection portions 5 and 5′ are inspected using a target substance (standard substance) with a known concentration. In the concentration ranges, the detection result of the target substance in the detection portions 5 and 5′ and the concentration of the target substance preferably have linearity. The molar amounts of the capture substances 8 and 8′ fixed to the detection portions 5 and 5′ can be determined through such an operation.

### (2) Examination of Condition of Preprocessing Portion 6′

Capture substances of various concentrations (100, 10, 1, 0.1, and 0.01 μg/mL) are prepared and fixed to the preprocessing portion 6′ of the micro-channel 2′ in the analysis device according to this embodiment. Next, the detectable concentration range of the detection portion 5′ is inspected using the standard substance. In this concentration range, the detection result of the target substance in the detection portion 5′ and the concentration of the target substance preferably have linearity.

### (3) Determination of Condition of Preprocessing Portion 6′

The concentration of the capture substance 3′ indicating a condition close to a desired concentration range is selected based on the result of the operation (2). Specifically, the concentration of the capture substance fixed to the preprocessing portion 6′ is selected so that the concentration range detectable in the detection portion 5′ includes at least a part of the concentration range confirmed in the operation (1) and detectable in the detection portion 5 and includes a higher concentration range which is not included in the concentration range detectable in the detection portion 5. The fixed condition of the preprocessing portion 6′ is determined by performing the operation (2) again using the capture substance with a concentration near the selected concentration.

For example, when the concentration of the target substance in the sample provided in the analysis falls is included in the concentration range of a to b (μg/mL), the fixed condition of the preprocessing portion is selected so that the concentration range detectable by the detection portion 5 is a range of x to b′ (μg/mL) and the concentration range detectable by the detection portion 5′ is a range of a′ to y (μg/mL) (where x≦a<a′≦b′<b≦y). A relation of “x=a” may be satisfied, but a relation of “x<a” is more preferable. A relation of “a′=b′” may be satisfied, but a relation of “a′<b′” is more preferable. A relation of “b=y” may be satisfied, but a relation of “b<y” is more preferable. Further, when x<<a, the preprocessing portion 6 may be provided in the micro-channel 2 and the concentration range detectable in the detection portion 5 may be moved toward a higher concentration side. In this case, it is necessary to fix the captured substance in which x does not exceed a.

In the analysis device according to this embodiment, as illustrated in FIG. 7(b), a value that regulates a flow direction of a fluid in the micro-channels 2 and 2′ from the injection portion 1 to the discharge portion 10 or controls flow of the fluid may be provided inside each micro-channel. For example, a valve 18 may be provided inside the micro-channel 2 between the injection portion 1 and the detection portion 5. A valve 18′ may be provided inside the micro-channel 2′ between the preprocessing portion 6′ and the detection portion 5′. In the analysis device according to this embodiment, although not illustrated, driving means for accelerating movement of the fluid in the micro-channels 2 and 2′ from the injection portion 1 to the discharge portion 10 may be connected to at least one of the injection portion 1 and the discharge portion 10.

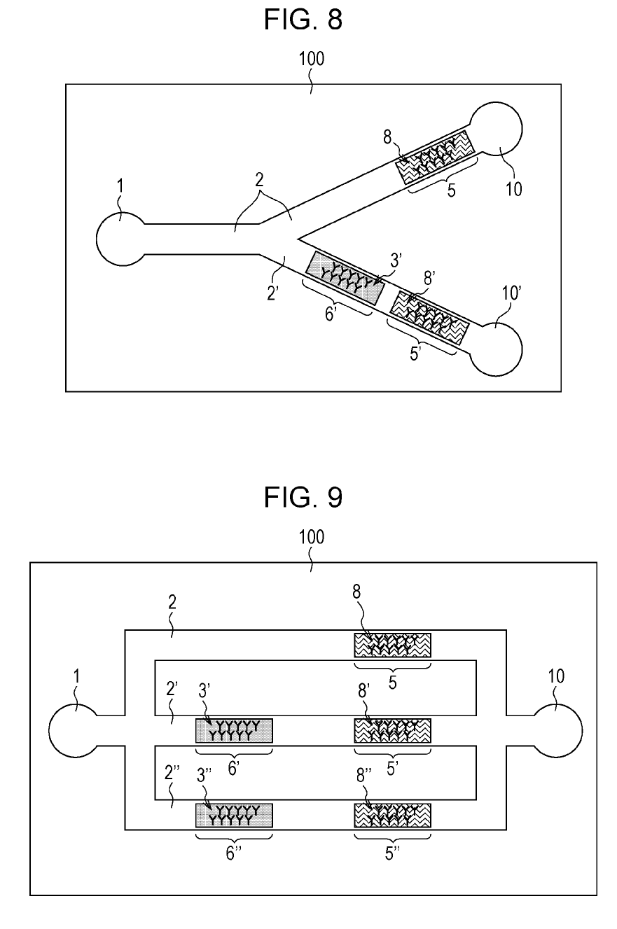

The configuration in which the single discharge portion is shared by the plurality of micro-channels has been illustrated in FIG. 7. However, as illustrated in FIG. 8, each of the plurality of micro-channels may be connected to each independent discharge portion. That is, in the micro-channel type analysis device according to this embodiment, as illustrated in FIG. 8, the micro-channel 2 that connects the injection portion 1 to the discharge portion 10 formed on the surface of the substrate 100 and the micro-channel 2′ that connects the injection portion 1 and a discharge portion 10′ may be formed on the surface of the substrate 100.

The configurations of the main constituents such as the substrate, the micro-channel, the injection portion, the discharge portion, the preprocessing portion, the detection portion, the valve structure, the driving means, and the like in the analysis device according to this embodiment are the same as those of the above-described first and second embodiments. Further, those skilled in the art who read the specification can appropriately modify the configuration of the first or second embodiment and also apply the method of preparing the capture substances, the measurement method, and the measurement result to this embodiment.

### Fourth Embodiment

As the specific configuration of the analysis device according to the third embodiment, the configuration in which the single bypass channel (the micro-channel 2′) is diverged from the micro-channel 2 which is the main flow path has been illustrated in FIGS. 7 and 8. However, the number of bypass channels is not particularly limited. Each bypass channel has a different detection range. Therefore, when the number of bypass channels increases, the detection range of the analysis device can be expanded.

As a micro-channel type analysis device according to a second embodiment, for example, an analysis device including three micro-channels can be exemplified, as illustrated in FIG. 9. In the analysis device, as illustrated in FIG. 9, three micro-channels 2, 2′, and 2″ are formed on the surface of a substrate 100. The micro-channel 2 connects the injection portion 1 to a discharge portion 10 on the surface of the substrate 100. The micro-channels 2′ and 2″ are bypass channels from which the micro-channel 2 is diverged and joined again between the injection portion 1 and the discharge portion 10. As illustrated in FIG. 9, the micro-channels 2′ and 2″ are diverged in the same division portion in the micro-channel 2 and are joined in the same joining portion.

Detection portions 5, 5′, and 5″ are provided inside the micro-channels 2, 2′, and 2″, respectively. Capture substances 8, 8′, and 8″ are fixed to the detection portions 5, 5′, and 5″. Preprocessing portions 6′ and 6″ are provided inside the micro-channels 2′ and 2″ between the injection portion 1 and the detection portions 5′ and 5″, respectively. Capture substances 3′ and 3″ are fixed to the preprocessing portions 6′ and 6″.

In the analysis device according to this embodiment, although not illustrated, a value that regulates a flow direction of a fluid in the micro-channels 2, 2′, and 2″ from the injection portion 1 to the discharge portion 10 or controls flow of the fluid may be provided inside each micro-channel.

In the analysis device according to this embodiment, although not illustrated, driving means for accelerating movement of the fluid in the micro-channels 2, 2′, and 2″ from the injection portion 1 to the discharge portion 10 may be connected to at least one of the injection portion and the discharge portion.

The configurations of the main constituents such as the substrate, the micro-channel, the injection portion, the discharge portion, the preprocessing portion, the detection portion, the valve structure, the driving means, and the like in the analysis device according to this embodiment are the same as those of the above-described first embodiment. Further, those skilled in the art who read the specification can appropriately modify the configuration of the first to third embodiments and also apply the method of preparing the capture substances, the measurement method, and the measurement result to this embodiment.

[Analysis Device According to Other Embodiments of the Invention]

In the analysis device according to the invention, each preprocessing portion preferably has a different preprocessing capability. The concentration range of the detection in the detection portion can be shifted using this preprocessing portion. Further, the “preprocessing capability” means an amount (for example, a molar amount) of a target substance to be reduced in the preprocessing portion, and specifically means the amount of the target substance captured by the capture substance disposed in the preprocessing portion. In order to prepare the preprocessing portions having different preprocessing capabilities, for example, the amount of capture substance disposed in each first preprocessing portion may be changed. That is, in the analysis device according to the invention, the amount of disposed capture substances are preferably different from each other in the first preprocessing portions in the plurality of first minute flow paths.

In the analysis device according to the invention, the detection sensitivities of the first detection portions in the plurality of first minute flow paths are preferably the same as each other. In the specification, the “detection sensitivity of the detection portion” means an amount (for example, a molar amount) of target substance detected in the detection portion, and specifically means the amount of target substance captured by the capture substance disposed in the detection portion. In this configuration, the concentration ranges of the detection in the detection portions can be shifted by using the preprocessing portions having the different preprocessing capabilities.

In the analysis device according to the invention, the plurality of first minute flow paths are preferably connected to the single discharge portion. The configuration of the analysis device is simpler when the configuration in which the single discharge portion is provided is realized rather than the configuration in which the plurality of discharge portions are provided. Therefore, the analysis device can be manufactured more easily. As the configuration in which the plurality of first minute flow paths are connected to the single discharge portion, a configuration can be exemplified in which each of the first minute flow paths is joined between the detection portion and the discharge portion.

In the analysis device according to the invention, the detection sensitivities of the first and second detection portions are preferably the same as each other. In this configuration, since the concentration of the target substance contained in the sample delivered to the first detection portion is reduced by the first preprocessing portion, the concentration range detectable in the first detection portion can be designed to be present to a concentration side higher than the concentration range detectable in the second detection portion.

In the analysis device according to the invention, a plurality of the first minute flow paths are preferably present, and first detection portions in the plurality of first minute flow paths preferably detect the substances at different concentration ranges, respectively. In this configuration, the calibration range of the analysis device can be expanded.

In the analysis device according to the invention, the preprocessing portions preferably have different preprocessing capabilities. The concentration range of the detection in the detection portion can be shifted by using this preprocessing portion. In order to prepare the preprocessing portions having different preprocessing capabilities, for example, the amount of capture substance disposed in each first preprocessing portion may be changed. That is, in the analysis device according to the invention, the amount of disposed capture substances are preferably different from each other in the first preprocessing portions in the plurality of first minute flow paths.

In the analysis device according to the invention, the detection sensitivities of the first detection portions in the plurality of first minute flow paths are preferably the same as each other. In this configuration, the concentration ranges of the detection in the detection portions can be shifted by using the preprocessing portions having the different preprocessing capabilities.

In the analysis device according to the invention, the first and second minute flow paths are preferably connected to the single discharge portion. The configuration of the analysis device is simpler when the configuration in which the single discharge portion is provided is realized rather than the configuration in which the plurality of discharge portions are provided. Therefore, the analysis device can be manufactured more easily. As the configuration in which the first and second minute flow paths are connected to the single discharge portion, a configuration can be exemplified in which the first and second minute flow paths are joined between the detection portion and the discharge portion.

In the analysis device according to the invention, the capture substances disposed in the respective detection portions are preferably the same substance and are preferably disposed under the same condition. In this configuration, the detection sensitivities of the respective detection portions can be the same as each other.

In the analysis device according to the invention, a valve structure is preferably provided inside each minute flow path. In the analysis device according to the invention, the above-described valve structure is preferably provided between the corresponding detection portion and preprocessing portion. When this configuration is used, a capture time of the target substance in the sample in the preprocessing portion can be arbitrarily ensured. Thus, the concentration of the substance analyzed in the detection portion can be further reduced.

In the specification, the term, “corresponding,” is used for two or more configurations functionally relevant to each other. For example, the corresponding detection portion and preprocessing portion” refer to the preprocessing portion that reduces the concentration of a specific substance and the detection portion that analyzes the specific substance with the concentration reduced by the preprocessing portion. For example, the preprocessing portion and the detection portion present in the same flow path correspond to each other. Further, “the corresponding injection portion and detection portion” may refer to a specific detection portion and the injection portion into which a sample containing a substance to be analyzed in this detection portion is injected (which corresponds to the introduction). “The corresponding injection portion and preprocessing portion” refer to a specific preprocessing portion and the injection portion into which a sample containing a substance to be reduced in this preprocessing portion is injected (which corresponds to the introduction).

In the analysis device according to the invention, the plurality of corresponding preprocessing portions are preferably provided between the corresponding injection portion and detection portion. The plurality of preprocessing portions may be disposed directly one another or may be disposed in parallel. When the plurality of preprocessing portions are disposed in parallel, at least one of the minute flow paths is configured to be diverged and joined again between the corresponding injection portion and detection portion, and thus the corresponding preprocessing portion is preferably provided in each of the plurality of provided divisions.

When this configuration is used, the plurality of preprocessing portions to which the capture substances capturing the target substance are fixed are present. Therefore, the concentration of the target substance can efficiently be reduced. In particular, when the parallel disposition is used, the concentration of the substance to be detected can be reduced in a shorter time by distributing the sample that contains the target substance into two or more samples and causing the distributed samples to pass through the preprocessing portions independently.

In the analysis device according to the invention, at least one of the preprocessing portions preferably includes a three-dimensional structure. This structure may be a pillar structure growing from a wall surface of the preprocessing portion, a porous structure, or a structure with a shape of a plurality of particles.

When this configuration is used, the three-dimensional structure is formed in the preprocessing portion. Therefore, the concentration of the substance to be detected can be more efficiently reduced. Thus, it is possible to obtain the advantage of shortening and integrating the analysis time. When the pillar structure is used, the area of the preprocessing portion stereoscopically increases. Therefore, the concentration of the detected substance can efficiently be reduced. When the porous structure is used, the area of the preprocessing portion stereoscopically increases. Therefore, the concentration of the detected substance can efficiently be reduced. When the structure with the shape of the plurality of particles is used, the area of the preprocessing portion stereoscopically increases. Therefore, the concentration of the detected substance can efficiently be reduced.

In the analysis device according to the invention, the capture substance capturing the substance to be detected is preferably an antibody corresponding to the substance to be detected. Further, the capture substance capturing the substance to be reduced is preferably an antibody corresponding to the substance to be reduced. Many substances to be detected by the micro-channel type analysis device used in the biochemical analysis are proteins in an organism. An antibody which is rarely degenerated is an optimum substance which is used as the capture substance.

In the analysis device according to the invention, detection means including a working electrode and a reference electrode is preferably provided in the detection portion. When this configuration is used, the target substance can be detected electrochemically in the detection portion. The target substance detected electrochemically may be a substance itself active electrochemically or may be a substance which is modified to a substance active electrochemically. As a method of detecting the target substance electrochemically, a current value obtainable from the substance active electrochemically may be measured by the detection means.

In the analysis device according to the invention, the detection portion is preferably formed of a transmissive material. When this configuration is used, the target substance can optically be detected in the detection portion. The optically detected target substance may be the target substance having optical characteristics or may be a substance which is modified to a substance having optical characteristics. Examples of the optical characteristics include light absorption characteristics, luminescence characteristics, and coloring characteristics. The luminescence includes fluorescence. Examples of the substance having the optical characteristics include light absorption pigments, luminescence pigments, and coloring pigments. A method of optically detecting the target substance may be a method of detecting the above-described optical characteristics. For example, a known method of the related art such as an ultraviolet-visible spectroscopic analysis method, a fluorescent analysis method, a chemical luminescent analysis method, or a thermal lens analysis method can be used as this method. When the target substance having the optical characteristics is used, quantitative measurement can be performed by measuring the optical characteristics (measuring a change in a chemical luminance amount, a change in fluorescence, and a change in absorbance).

In the analysis device according to the invention, additional detection means is preferably provided in the preprocessing portion. When this configuration is used, the target substance can be detected in the preprocessing portion. For example, when the detection means includes a working electrode and a reference electrode, the detection means can detect a substance active electrochemically.

In the analysis device according to the invention, the preprocessing portion is preferably formed of a transmissive material. When this configuration is used, the optical detection can be performed in the preprocessing portion. Therefore, the quantitative measurement can be performed by measuring a change in fluorescence or absorbance.

The sample to be applied to the analysis device according to the invention is preferably blood and the substance to be detected is a blood component. Examples of the blood component include a plasma protein, a lipoprotein, a secretory protein, a hormone, a complement, or sugar. When this configuration is used, the component (for example, a plasma protein, a lipoprotein, a secretory protein, a hormone, a complement, or sugar in blood) in blood can be analyzed as the detection target substance.

An analysis method according to the invention includes quantitatively measuring a concentration of a target substance in a sample provided in analysis without diluting the sample using the analysis device according to the invention. The content of the sample used in the micro technology is very small. When a complicated dilution operation is included at the time of preparing a small amount of sample, an error occurs in every preparation. Therefore, it is difficult to perform accurate analysis, and thus repeatability and/or reliability of the analysis may deteriorate. In this configuration, since the sample dilution operation can be omitted, the reproducibility and/or reliability of the analysis can be improved. Further, the quantitative measurement can be performed even when the user does not perform the dilution operation.

In the analysis method according to the invention, the target substance is preferably a component in blood. In particular, since a danger of affection to an infection disease of the user is involved in order to operate blood, an extreme caution is necessary in the treatment. By simplifying the process of preparing the sample, such a danger can be reduced. When this configuration is used, the sample dilution operation can be omitted. Therefore, the user can treat the blood sample more safely and easily.

Preferably, the analysis method according to the invention includes determining a concentration of a substance based on a concentration of a substance detected by each detection portion and a concentration range detectable by each detection portion. Thus, by considering the concentration of the target substance detected in each detection portion and the concentration range detectable in each detection portion, the concentration of the target substance detected out of the concentration range detectable in the detection portion can be determined to be “incorrect” and the concentration of the target substance detected within the concentration range detectable in the detection portion can be determined to be a “correct concentration.” By performing this determination, the accurate concentration of the target substance in the sample can be determined.

Preferably, the analysis method according to the invention includes determining whether an analysis error occurs based on the concentration of the substance detected by each detection portion and the concentration range detectable by each detection portion. In this configuration, when the concentration of the target substance is not detected within the concentration range detectable in the detection portion and the concentration of the target substance is detected out of the concentration range detectable in the detection portion, it can be determined that the detection fails. Further, when the concentration of the target substance can be detected within the concentration range detectable in the detection portion, it can be determined that the detection succeeds.

Preferably, the analysis method according to the invention includes measuring whether the target substance is captured in each preprocessing portion and determining that the analysis error occurs, when a given amount of target substance is not captured in the preprocessing portion. When this configuration is used, not only the quantitative measurement can be performed in the detection portion, but the quantitative measurement can also be performed in the preprocessing portion likewise. When a desired molar amount is not captured in the preprocessing portion, it can be determined that the detection fails. For example, it is effective to know that the activity of the capture substance deteriorates.

Hereinafter, the embodiments of the invention will be described in more detail by giving examples.

### EXAMPLES

### Example 1

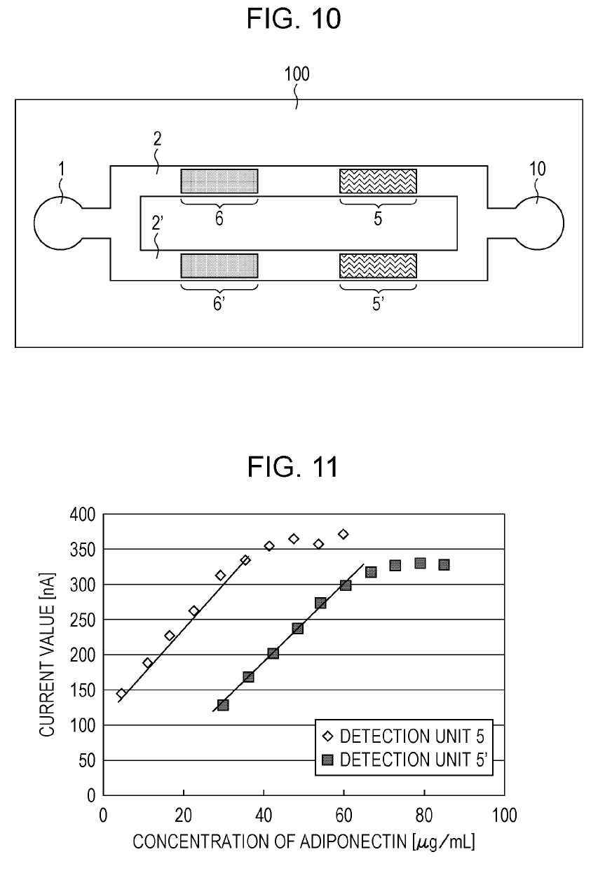

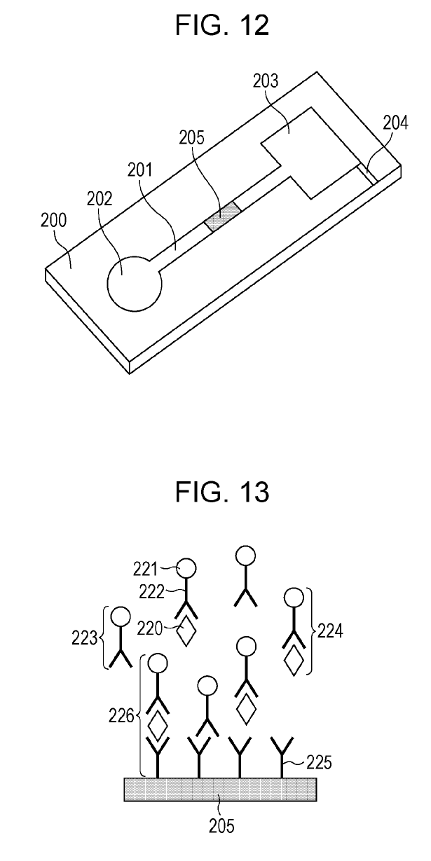

In Example 1, a micro-channel type analysis device (micro-channel chip) illustrated in FIG. 10 was manufactured as follows. That is, a micro-channel 2 was formed on a substrate 100 of PDMS (POLYDIMETHYLSILOXANE, Dow Corning Toray Co., Ltd). An injection portion 1 and a discharge portion 10 were formed to be connected to both ends of the micro-channel 2. A detection portion 5 was formed inside the micro-channel 2. A preprocessing portion 6 was provided inside the micro-channel 2 between the injection portion 1 and the detection portion 5.

Next, a micro-channel 2′ was formed on the substrate 100. Specifically, the micro-channel 2′ was formed so as to be diverged from the micro-channel 2 in a division portion between the injection portion 1 and the preprocessing portion 6 and be joined with the micro-channel 2 in a joining portion between the detection portion 5 and the discharge portion 10. Then, the detection portion 5′ was provided inside the micro-channel 2′ between the division portion and the joining portion. Further, a preprocessing portion 6′ was provided inside the micro-channel 2′ between the division portion and the detection portion 5′.

Two through holes (not illustrated) penetrating the substrate 100 communicated with the injection portion 1 and the discharge portion 10. Further, dammed portions (not illustrated) were provided inside the preprocessing portions 6 and 6′.

Detection electrodes (not illustrated) including a working electrode, a reference electrode, and a counter electrode were formed in the detection portions 5 and 5′ using a photolithographic method. Specifically, gold electrodes (the working electrodes and the counter electrodes) were formed by forming resist patterns on the micro-channels 2 and 2′ and sputtering titanium and then gold. Further, silver/silver chloride electrodes (the reference electrodes) were formed by sputtering titanium and then silver and performing a chloride process.

The width, the length, and the depth of the micro-channel 2 were 600 μm, 2000 μm, and 50 μm, respectively. The width, the length, and the depth of the micro-channel 2′ were 600 μm, 2000 μm, and 50 μm, respectively. The working electrode had a length of 200 μm×a width of 600 μm. The counter electrode had a length of 200 μm×a width of 600 μm. The reference electrode had a length of 50 μm×a width of 50 μm.

Next, a carboxyl group was introduced to the gold surface on the detection electrode using the thio SAM solution (11-Mercaptoundecanoic acid (Dojindo Laboratories):6-hydroxy-1-hexanethiol (Dojindo Laboratories)=1:9) of 10 mM. Thereafter, the carboxyl group was activated using 1-ethyl-3-[(3-dimethylamino) propyl]carbodiimide hydrochloride (EDC) of 100 mg/mL and N-hydroxysulfosuccinimide of 100 mg/mL, and a solution of an adiponectin antibody (R&D System Co., Ltd) of 100 ng/mL reacted with the carboxyl group for 30 minutes. Thus, the adiponectin antibody was fixed to the detection electrode. Then, a solution containing the unreacted adiponectin antibody was removed. After the adiponectin antibody was fixed, a PDMS substrate (not illustrated) serving as a cover was adhered to a PDMS substrate in which the micro-channels 2 and 2′ were formed so that the micro-channels 2 and 2′ were covered.

Magnetic microparticles with a diameter of 15 μm and the adiponectin antibody (R&D System Co., Ltd) of 10 μg/mL were mixed and incubated at 37° C. for 1 hour. Thus, the adiponectin antibody was fixed to the magnetic microparticles. The magnetic microparticles were sufficiently cleaned with PBS containing Tween 20 of 0.05%. A solution of 5 μL containing the magnetic microparticles of 1% (w/v) after the cleaning was injected from the injection portion 1 to the inside of the micro-channel 2 and was carried up to the position of the preprocessing portion 6 using a magnet. Further, the solution of 50 μL containing the magnetic microparticles was injected from the injection portion 1 to the inside of the micro-channel 2′ and was carried up to the position of the preprocessing portion 6′ using a magnet. The solution containing the magnetic microparticles was moved inside the micro-channels 2 and 2″ by connecting the discharge portion 10 to a suction pump and using the suction portion, and then the magnetic microparticles were dammed in the dammed portion. Thus, the preprocessing portions 6 and 6′ formed of the magnetic microparticles were manufactured.

Further, the blocking in the micro-channels 2 and 2′ was performed using Protein-Free (Thermo Co., Ltd) which is a non-specific absorption inhibitor.

In this way, the micro-channel type analysis device analyzing the adiponectin was manufactured.

A standard adiponectin solution with an adiponectin concentration of 4.6 μg/mL, 10.8 μg/mL, 16.9 μg/mL, 23.0 μg/mL, 29.2 μg/mL, 29.6 μg/mL, 35.3 μg/mL, 35.8 μg/mL, 41.4 μg/mL, 41.9 μg/mL, 47.5 μg/mL, 48.0 μg/mL, 53.7 μg/mL, 54.2 μg/mL, 59.8 μg/mL, 60.3 μg/mL, 66.4 μg/mL, 72.5 μg/mL, 78.7 μg/mL, or 84.8 μg/mL was prepared. The adiponectin in the standard adiponectin solution was detected by applying the prepared standard adiponectin solutions to the above-described analysis device one by one.

Specifically, the adiponectin was detected as follows. The standard adiponectin solution of 5 μL was injected from the injection portion 1 to the insides of the micro-channels 2 and 2′. This solution was moved up to the detection portions 5 and 5′ using the suction pump and was stopped on the detection portions 5 and 5′ for 3 minutes. Thus, the adiponectin in this solution was bonded with the adiponectin antibody on the detection portions 5 and 5′. Thereafter, the standard adiponectin solution was discharged from the micro-channels 2 and 2′ using the suction pump.

Next, the PBS was injected from the injection portion 1 to the insides of the micro-channels 2 and 2′ and the insides of the micro-channels 2 and 2′ were sufficiently cleaned. A solution containing an ALP-modified adiponectin antibody of 1 μg/mL produced using the alkaline phosphatase (ALP) Labeling Kit (Dojindo Laboratories) was injected from the injection portion 1 to the insides of the micro-channels 2 and 2′. The solution containing the ALP-modified adiponectin antibody was moved up to the detection portions 5 and 5′ using the suction pump and was stopped on the detection portions 5 and 5′ for 3 minutes. Thus, the ALP-modified adiponectin antibody in the solution was bonded with the adiponectin captured on the detection portions 5 and 5′. Thereafter, the solution containing the unreacted ALP-modified adiponectin antibody was discharged from the micro-channels 2 and 2′ using the suction pump. A glycine NaOH buffer (pH 9.0) was injected from the injection portion 1 to sufficiently clean the insides of the micro-channels 2 and 2′.

Next, a solution of a para-aminophenylphosphate of 1 mM was injected from the injection portion 1 to the insides of the micro-channels 2 and 2′, was moved up to the detection portions 5 and 5′ using the suction pump, and was stopped on the detection portions 5 and 5′ for 3 minutes. Thus, the para-aminophenylphosphate reacted with the ALP to generate a current. The peak current value (nA) of the generated current was detected by the detection portions 5 and 5′.

As illustrated in FIG. 11, the detection portion 5 detected currents of 144 nA, 189 nA, 227 nA, 264 nA, 312 nA, 336 nA, 355 nA, 364 nA, 358 nA, and 372 nA, when the adiponectin concentrations of the standard adiponectin solution were 4.6 μg/mL, 10.8 μg/mL, 16.9 μg/mL, 23.0 μg/mL, 29.2 μg/mL, 35.3 μg/mL, 41.4 μg/mL, 47.5 μg/mL, 53.7 μg/mL, and 59.8 μg/mL, respectively. The detection portion 5′ detected currents of 129 nA, 170 nA, 204 nA, 238 nA, 275 nA, 300 nA, 320 nA, 328 nA, 332 nA, and 330 nA, when the adiponectin concentrations of the standard adiponectin solution were 29.6 μg/mL, 35.8 μg/mL, 41.9 μg/mL, 48.0 μg/mL, 54.2 μg/mL, 60.3 μg/mL, 66.4 μg/mL, 72.5 μg/mL, 78.7 μg/mL, and 84.8 μg/mL, respectively. The calibration curves were produced from the values of the detected currents.

The graph of the calibration curves are illustrated in FIG. 11. In FIG. 11, the adinponectin with concentrations of 1 μg/mL to 35 μg/mL could be quantitatively measured in the detection portion 5, and the adinponectin with concentrations of 30 μg/mL to 65 μg/mL could be quantitatively measured in the detection portion 5′. That is, the calibration range of the adinponectin of the detection portion 5 is a range of 1 μg/mL to 35 μg/mL and the calibration range of the adinponectin of the detection portion 5′ is a range of 30 μg/mL to 65 μg/mL. Accordingly, the calibration range of the adinponectin of the analysis device manufactured in this example is 1 μg/ml, to 65 μg/mL. For example, when the concentration of the adinponectin contained in the sample is in the range of 1 μg/mL to 65 μg/mL, the concentration of the adinponectin can be measured using this analysis device in spite of the fact that any sample is used.

### INDUSTRIAL APPLICABILITY

When the analysis device according to the present invention is used, the concentration of a small amount of sample can be measured without performing a dilution operation. Therefore, the invention can be applied to fields in which a micro-channel chip, such as a chemical micro-device (for example, a micro-channel chip and a microreactor) used to detect a minute chemical substance and a biosensor (for example, an allergen sensor), that uses a μ-TAS technology is used.

### REFERENCE SIGNS LIST

1 injection portion
2 micro-channel
3 capture substance
4 air hole
5 detection portion
6 preprocessing portion
6a pillar structure
6b minute particle
8 capture substance
9 dammed portion
10 discharge portion
13 capture substance
16 preprocessing portion
18 valve
100 substrate
101 cover

1.-35. (canceled)

36. An analysis device comprising:
a plurality of first minute flow paths that connect an injection portion accommodating a fluid to be injected to a discharge portion discharging the fluid,
wherein the plurality of first minute flow paths are connected to the single injection portion,
wherein in each of the plurality of first minute flow paths, a first detection portion is provided between the injection portion and the discharge portion and a first preprocessing portion reducing a concentration of a substance in the fluid is further provided between the injection portion and the first detection portion,
wherein a capture substance capturing a substance to be detected is disposed in the first detection portion,
wherein a capture substance capturing a substance to be reduced is disposed in the first preprocessing portion,
wherein substances to be detected in the first detection portions of the plurality of first minute flow paths are the same as each other,
wherein the first detection portions in the plurality of first minute flow paths detect the substances in different concentration ranges, respectively, and
wherein the substance to be detected is the same as the substance to be reduced.

37. The analysis device according to claim 36, wherein amounts of the disposed capture substances are different from each other in the first preprocessing portions in the plurality of first minute flow paths.

38. The analysis device according to claim 36, wherein detection sensitivities of the first detection portions in the plurality of first minute flow paths are the same as each other.

39. An analysis device comprising:
first and second minute flow paths that connect an injection portion accommodating a fluid to be injected to a discharge portion discharging the fluid,
wherein the first and second minute flow paths are connected to the single injection portion,
wherein first and second detection portions are provided in the first and second minute flow paths, respectively,
wherein a first preprocessing portion reducing a concentration of a substance in the fluid is further provided in the first minute flow path between the injection portion and the first detection portion,
wherein a first capture substance capturing a substance to be detected is disposed in the first detection portion,
wherein a second capture substance capturing the substance to be detected is disposed in the second detection portion,
wherein a capture substance capturing the substance to be reduced is disposed in the first preprocessing portion,
wherein the substances to be detected in the first and second detection portions are the same as each other,
wherein the first and second detection portions detect the substances in different concentration ranges, respectively, and
wherein the substance to be detected is the same as the substance to be reduced.

40. The analysis device according to claim 39, wherein detection sensitivities of the first and second detection portions are the same as each other.

41. The analysis device according to claim 39, wherein a plurality of the first minute flow paths are present, and first detection portions in the plurality of first minute flow paths detect the substances at different concentration ranges, respectively.

42. The analysis device according to claim 41, wherein in first preprocessing portions in the plurality of first minute flow paths, amounts of the disposed capture substances are different from each other.

43. The analysis device according to claim 41, wherein detection sensitivities of the first detection portions in the plurality of first minute flow paths are the same as each other.

44. The analysis device according to claim 36, wherein the capture substances disposed in the detection portions are the same substances and are disposed under the same condition.

45. The analysis device according to claim 36, wherein additional detection means is provided in the preprocessing portion.

46. The analysis device according to claim 36, wherein the preprocessing portion is formed of a transmissive material.

47. The analysis device according to claim 36, wherein the plurality of corresponding preprocessing portions are provided between the corresponding injection portion and detection portion.

48. The analysis device according to claim 47, wherein at least one of the minute flow paths is configured to be diverged and joined again between the corresponding injection portion and detection portion, and the corresponding preprocessing portion is provided in each of the plurality of provided divisions.

49. The analysis device according to claim 36, wherein at least one of the preprocessing portions includes a three-dimensional structure, and
wherein the structure is a porous structure.

50. The analysis device according to claim 36, wherein at least one of the preprocessing portions includes a three-dimensional structure, and
wherein the structure is a structure with a shape of a plurality of particles.

51. The analysis device according to claim 36, wherein the detection portion is formed of a transmissive material.

52. An analysis method comprising:
quantitatively measuring a concentration of a target substance in a sample provided in analysis without diluting the sample using the analysis device according to claim 36.

53. The analysis method according to claim 52, further comprising:
determining whether an analysis error occurs based on the concentration of the substance detected by each detection portion and the concentration range detectable by each detection portion.

54. The analysis method according to 52, further comprising:
measuring whether the target substance is captured in each preprocessing portion, and determining, when a given amount of target substance is not captured in the preprocessing portion, that the analysis error occurs.
